(19) United States
(12) Patent Application Publication
(10) Pub. No.: US 2022/0221930 A1
(43) Pub. Date: Jul. 14, 2022

(54) ELECTRONIC DEVICE AND OPERATION METHOD OF ELECTRONIC DEVICE

(71) Applicant: Samsung Electronics Co., Ltd., Gyeonggi-do (KR)

(72) Inventors: Hyunjoo KIM, Gyeonggi-do (KR); Boram BAE, Gyeonggi-do (KR); Taehwan SON, Gyeonggi-do (KR); Guhyun YANG, Gyeonggi-do (KR); Jungmi KIM, Gyeonggi-do (KR)

(21) Appl. No.: 17/671,992

(22) Filed: Feb. 15, 2022

Related U.S. Application Data
(63) Continuation of application No. PCT/KR2022/000569, filed on Jan. 12, 2022.

(30) Foreign Application Priority Data
Jan. 12, 2021 (KR) 10-2021-0004181

(51) Int. Cl. G06F 3/01; H04N 5/232; G06T 7/246

(52) U.S. Cl. CPC G06F 3/011; G06T 2207/30196; G06T 7/246; H04N 5/23219

(57) ABSTRACT

An electronic device and method are disclosed. The electronic device includes a display, a camera, a memory and a processor. The processor implements the method, including: analyze an image of the content to generate corrective posture information, capture an image including a first user via the camera while displaying the content, analyze the captured image including the first user to generate first posture information of the first user, generate first feedback information, by comparing the corrective posture information to the first posture information, select a first external electronic device for transmission of the first feedback information, based on one of a property of the first feedback information, a function of the first external electronic device, a number of users, and a stored relationship of the user to the first external electronic device, and transmit the first feedback information to the selected first external electronic device.

### CROSS-REFERENCE TO RELATED APPLICATION(S)

This application is a continuation of International Application No. PCT/KR2022/000569, filed on Jan. 12, 2022, which claims priority to Korean Patent Application No. 10-2021-0004181, filed on Jan. 12, 2021 in the Korean Intellectual Property Office, the disclosures of which are herein incorporated by reference.

### TECHNICAL FIELD

Certain embodiments disclosed herein relate to an electronic device and an electronic device operating method and, more specifically, to an electronic device and an electronic device operating method, wherein feedback of a user motion is provided with regard to an object motion inside content.

### BACKGROUND

As electronic device communication and user interface technology are developed and advanced, users are better able to access a diverse quantity of information using these electronic devices without spatial and temporal restrictions.

Users may acquire various types data, such as pictures, texts, sounds, and moving images, from external media sources. Particularly, users may easily acquire multimedia and video as video sharing platforms have grown in popularity.

Many exercise-related videos (e.g., home training videos) are uploaded to video sharing platforms, enabling easy access to the same. Accordingly, users may perform physical exercise by watching and imitating movements within the videos, without help from professionals (e.g., personal trainers).

However, there is a problem in that exercise videos acquired from sharing platforms typically fail to provide detailed information on the exercise motions performed therein, and likewise, users do not receive any feedback on whether their own motions are accurate, increasing the likelihood of injury.

With the widespread proliferation of video sharing platforms, users of electronic devices have access to videos ranging on a wide variety of topics, such as exercise, education, gaming, cooking, etc.

### SUMMARY

Certain embodiments disclosed herein may enable analysis of content included in the shared videos, configuration of associated images, and determination of accuracy of motions associated with the shared video acquired from an external media source (e.g., a third-party application), enabling provision of motion assistance and guidance information.

Specifically, an electronic device according to certain embodiments disclosed herein may analyze an acquired video so as to distinguish sections including motions that a user may imitate (e.g., exercise, dance, or the like performed by someone in the video), classify the motions according to a number of preset types, and combine functionality with other connected external electronic devices, thereby facilitating more accurate reproduction of the depicted motions by a user.

For example, an electronic device disclosed herein may analyze an exercise video uploaded to an external media source, distinguish sections of the video according to the type of exercises depicted therein, and utilize this information to interoperate with other external electronic devices available to the user to enable more accurate imitation of the motions. The information may include descriptions of the exercise motion, a count of appropriate number of repetitions, determinations of error in the user's form and/or motion, etc. The external devices may include sensors capable of tracking motions, image capture devices such as smartphones, wearable devices that can measure the user's motion, audio output devices that can output provide audio guides, etc. Thus, a user interface operating in one or multiple formats may be capable of supporting the user's exercise regimen. For example, a user interface (UI) may display a count of repetitions and a total exercise time through textual output, and movement information may likewise be displayed in a section centered on a particular exercise motion. Therefore, the user may benefit by exercising while viewing the shared exercise media received from an external media source, and also view media capture of themselves, without using an external exercise assist application. The user may be provided with feedback on the accuracy of their motions, the count of their repetitions, in addition to receiving biometric tracking, such as heartbeat and caloric information, as measured by this or another electronic device.

The invention thus addresses technical issues relevant to operation of electronic devices and increases user convenience in operating of the same.

Technical problems to be solved herein are not limited to the above-mentioned technical problems, and other technical problems not mentioned herein will be clearly understood from the following description by those skilled in the art to which the disclosure pertains.

An electronic device according to certain embodiments disclosed herein may include: a display; a camera; a memory configured to store content, and a processor operatively coupled to the display, the camera, and the memory, wherein the processor is configured to: analyze at least one image of the content to generate corrective posture information from an object depicted in the at least one image, capture an image including a first user via the camera while displaying the content, analyze the captured image including the first user to generate first posture information of the first user, generate first feedback information, by comparing the corrective posture information to the first posture information, select a first external electronic device for transmission of the first feedback information, based on one of a property of the first feedback information, a function of the first external electronic device, a number of users, and a stored relationship of the user to the first external electronic device, and transmit the first feedback information to the selected first external electronic device.

A method of operating an electronic device according to certain embodiments disclosed herein may include: analyzing at least one image included in a stored content to generate corrective posture information of an object depicted in the at least one image, capturing an image including a first user via a camera while displaying the stored content on a display, analyzing the captured image to generate first posture information of the first user, generating first feedback information by comparing the corrective posture information to the generated first posture information, selecting a first external electronic device for transmission of the generated feedback information, based on one of a property of the first feedback information, a function of the first external electronic device, a number of users, and a stored relationship between the first user to the first external electronic device, and transmitting the generated feedback information to the selected external electronic device.

A user may more accurately perform exercises depicted in share exercise videos, without assistance from a personal trainer, because of the analysis and provision of exercise motion guidance and assistance information.

In addition, the user may be provided with exercise assistance information with no content limit by selecting a video including a desired motion/exercise from an external media source, and thereby receiving motion assistance information.

In addition, the user may be provided with feedback regarding the accuracy of their movements, enabling correction of the posture and motion, thereby improving the benefit from exercise and preventing injury.

In addition, exercise assistance information may be divided for provision to a number of external electronic devices having different user-machine interfaces and components therein, based on attributes of the exercise assistance information and/or the particular function of the external electronic device, thereby providing a cohesive multi-device environment for exercise.

In addition, if multiple users are present, each user may be provided with exercise assistance information specific to themselves, allowing for provision of user-customized assistance information.

### BRIEF DESCRIPTION OF DRAWINGS

In connection with the description of the drawings, the same or similar reference numerals may be used for the same or similar components.

FIG. 1 is a block diagram of an electronic device in a network environment, according to certain embodiments of the disclosure.

FIG. 2 is a block diagram of an electronic device according to certain embodiments disclosed herein.

FIG. 3 is a flowchart illustrating a method in which a processor controls an electronic device to provide feedback of a user's motion with respect to an object motion in content, according to certain embodiments of the disclosure.

FIG. 4 is a diagram illustrating an electronic device according to certain embodiments described in this document and an external electronic device related to the electronic device.

FIGS. 5A and 5B are diagrams illustrating examples of content screens, according to certain embodiments described herein.

FIGS. 5C and 5D are diagrams illustrating an example of a user screen including a user, according to certain embodiments described herein.

FIGS. 5E and 5F are diagrams illustrating examples of displaying a content screen and a user screen, according to certain embodiments.

FIG. 6 is a diagram illustrating an example of a second external electronic device connected to an electronic device, according to certain embodiments of the disclosure.

FIGS. 7A, 7B, and 7C are diagrams illustrating examples in which a processor controls an external electronic device to output various information from the external electronic device, according to certain embodiments of the disclosure.

FIGS. 8A, 8B, 8C, 8D, and 8E are diagrams illustrating an example of a UI included in an electronic device according to certain embodiments disclosed in this document.

### DETAILED DESCRIPTION

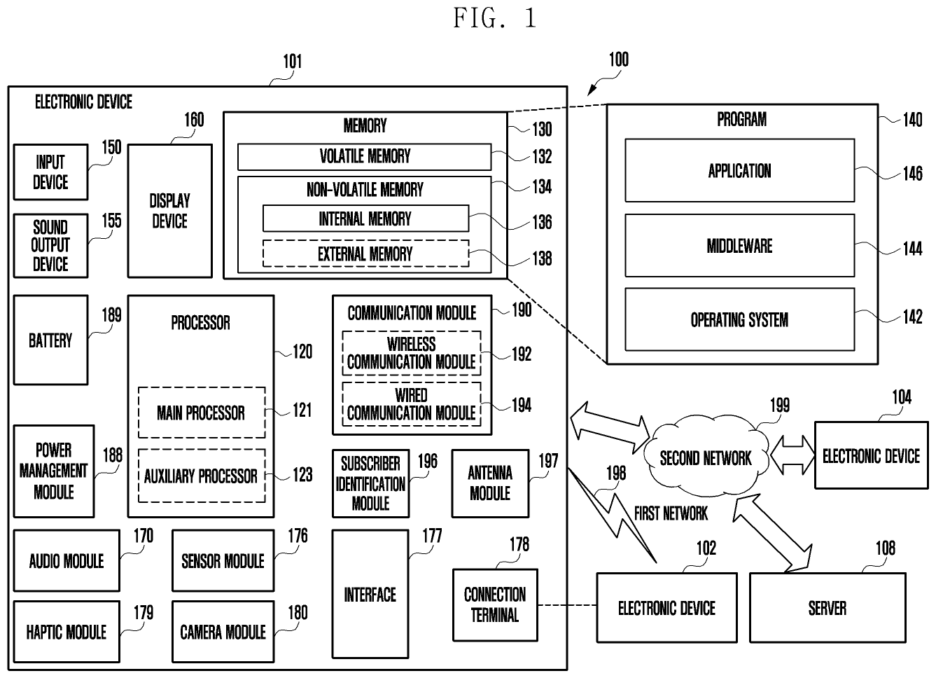

FIG. 1 is a block diagram illustrating an electronic device 101 in a network environment 100 according to certain embodiments. Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module(SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to an embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted Boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thererto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element implemented using a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to certain embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra-low-latency services using, e.g., distributed computing or mobile edge computing. In another embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

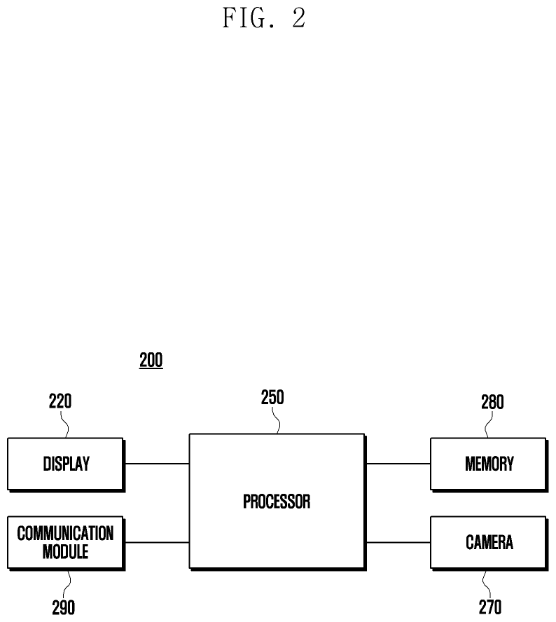

FIG. 2 is a block diagram of an electronic device according to certain embodiments disclosed herein.

Referring to FIG. 2, the electronic device (e.g., the electronic device 101) 200 may include a processor (e.g., the processor 120 of FIG. 1) 250, a memory (e.g., the memory 130 of FIG. 1) 280, a display (e.g., the display 160 of FIG. 1) 220, a communication module (the communication module 190 of FIG. 1), and/or a camera (e.g., the camera module 180 of FIG. 1) 270. The components included in FIG. 2 are for some of the components included in the electronic device 200, and the electronic device 200 may include various other components as shown in FIG. 1.

The memory 280 may be the memory 130 described with reference to FIG. 1. The memory 280 may temporarily or non-temporarily store at least one of content received from the outside and/or content stored in another memory of the electronic device 200 and/or in an external memory physically connected to the electronic device 200. Here, the content may include at least one of image information, audio information, and text.

The display 220 may be the display module 160 described with reference to FIG. 1. The processor 250 may be connected to the display 220 to process information so that various information may be visually displayed through the display 220.

The communication module 290 may be the communication module 190 described with reference to FIG. 1. The communication module 290 may receive and/or transmit various information by communicating with an external electronic device through a network (e.g., the first network 198 and/or the second network 199 of FIG. 1). The processor 250 may be connected to the communication module 290 to process various information received by the communication module 290 from an external electronic device. Furthermore, the processor 250 may control the communication module 290 to transmit various information to an external electronic device.

The camera 270 may photograph the environment around a user and/or the electronic device 200. The processor 250 may be connected to the camera 270 to process various image information photographed by the camera 270.

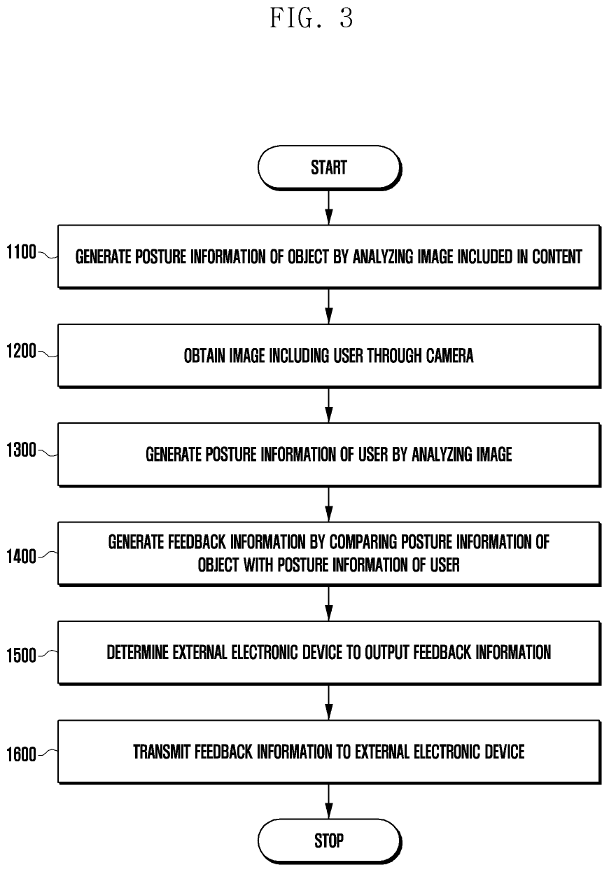

FIG. 3 is a flowchart illustrating a method in which a processor controls an electronic device to provide feedback of a user's motion with respect to an object motion in content, according to certain embodiments of the disclosure;

According to certain embodiments, in operation 1100, a processor (e.g., the processor 250 of FIG. 2) may analyze the image included in the content to generate posture information of the object included in the image.

According to an embodiment, the content may include various images (e.g., exercise images for home training) obtained from an external media source. The content may include at least one of image information, audio information, and text.

According to an embodiment, the processor 250 may distinguish a background area and an object performing a motion within the content, such as a trainer depicted in the video. According to an embodiment, the posture information of the object may mean relative position information of each part of the body of the object performing a motion.

According to an embodiment, the processor 250 may input an image frame of the image included in the content to a learned artificial intelligence model to obtain a feature value of the input image frame, and generate the posture information of the object.

According to certain embodiments, the processor 250 may analyze the image included in the content to generate motion information (e.g., division of motion, name of motion) of the object included in the image. According to an embodiment, the processor 250 may generate section information by dividing the motion section of the content, based on the motion information of the object. For example, the processor 250 may divide a section in which the object performs a first motion as a first section and a section in which the object performs a second motion as a second section. The processor 250 may generate information on a start position, an end position, and a length of each section in the content as section information of each section. According to an embodiment, the processor 250 may determine the action name based on the action information of the object. For example, in response to the object performing the first action, the processor 250 may match a general action name (e.g., jumping lunge, wide squat, burpee test, crunch, dance) of the first action. According to an embodiment, the processor 250 may obtain a feature value of the input image frame by inputting an image frame of the image included in the content to the learned artificial intelligence model, and generate motion information of the object, based on the obtained feature value.

According to certain embodiments, in operation 1200, the processor 250 may obtain an image including a depiction of the user through the camera.

According to an embodiment, the camera by which the processor 250 obtains the image including the user may include a camera (e.g., the camera 270 of FIG. 2) included in the electronic device 200.

According to an embodiment, the camera by which the processor 250 obtains the image including the user may include a camera (e.g., a webcam, a camcorder, a wireless camera) provided in an external electronic device, and the camera provided in the external electronic device may be connected to the electronic device 200 by wire and/or wirelessly.

According to an embodiment, the processor 250 may control the camera to operate in a shooting mode while content is output, and the processor 250 may obtain the image including the user photographed by the camera.

According to certain embodiments, in operation 1300, the processor 250 may analyze the image including the user to generate posture information of the user.

According to an embodiment, the processor 250 may distinguish the background area from the user in the image photographed by the camera. The posture information of the user may mean relative position information of each part of the user's body in the image. According to an embodiment, the processor 250 may input an image frame of an image photographed by the camera to the learned artificial intelligence model to obtain a feature value of the input image frame, and may generate posture information of the user, based on the obtained feature value.

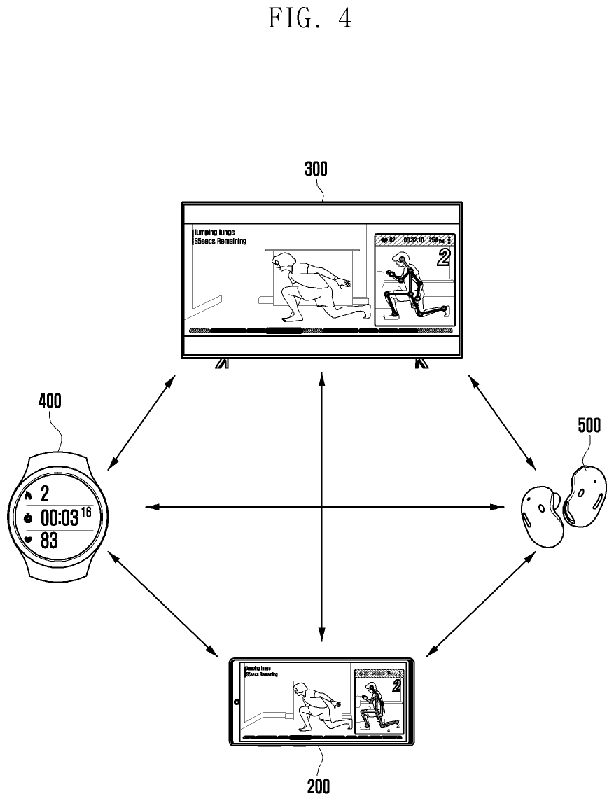

According to certain embodiments, the processor 250 may obtain biometric information (e.g., heart rate and/or calories) and/or motion information (e.g., motion) of the user received from an external device (e.g., the second external device 400 of FIG. 4) by the communication module 290.

According to certain embodiments, in operation 1400, the processor 250 may compare the posture information of the object (e.g., “corrective” posture information of the trainer) with the posture information of the user, and may generate feedback information to be provided to the user, based on the comparison result.

According to an embodiment, the processor 250 may determine a similarity between the posture information of the object and the posture information of the user based on the feedback information, and may divide the posture of the user into at least two or more regions based on the similarity. For example, the processor 250 may subdivide the user's body into a plurality of regions, and determine whether each region is a matching region (e.g., a region where the similarity is greater than or equal to a first value), a similar region (e.g., a region in which the similarity is less than the first value and greater than or equal to a second value), and/or a dissimilar region (e.g., a region in which the similarity is less than the second value), based on the similarity.

According to an embodiment, the processor 250 may output the feedback information in a visualized form. According to an embodiment, the processor 250 may implement the feedback information as guidance visualized in the form of a figure. For example, the processor 250 may generate nodes corresponding to certain parts of the user's body (e.g., joints such as wrists, elbows, head, pelvis, and popliteal muscles), and add lines connecting the nodes to generate the abstract form of the human body, and may output the posture of the user using the abstract humanoid visualization. Further, the colors of lines connecting between nodes may be set differently depending on whether the user's posture within the subregion is classified as “matching,” “similar” or “dissimilar” (e.g., a first color line for the matching region, a second color line for the similar region, and a third color line for the dissimilar region) in response to the information that divides the posture of the user into the matching region, the similar region, and the dissimilar region, based on the similarity.

According to an embodiment, the processor 250 may implement the feedback information in the form of audio. For example, the processor 250 may implement a sentence guiding a posture in the form of audio in order to increase the similarity between the posture information of the user and the posture information of the object in response to the dissimilar region.

According to certain embodiments, in operation 1500, the processor 250 may select an external electronic device for output of the feedback information. For example, a plurality of external devices may be communicatively available. According to an embodiment, the processor 250 may determine the external electronic device to output the feedback information, based on at least one of the attributes of the feedback information, a prestored function of the external electronic device, the present number of users, and a prestored relationship between the user and the external electronic device.

According to an embodiment, the processor 250 may determine to output the feedback information to an external electronic device equipped with a sound output function, in response to implementing of the feedback information in an audio format. According to an embodiment, the processor 250 may determine to output the feedback information to an external electronic device including a display, in response to implementing of the feedback information in a visualization form.

According to an embodiment, the processor 250 may determine to output first feedback information corresponding to a first user to an external electronic device of the first user and to output second feedback information corresponding to a second user to an external electronic device of the second user, in response to the generation of the plurality of pieces of feedback information for the plurality of users.

According to certain embodiments, the processor 250 may determine an external electronic device to output motion information of the object in the content and/or biometric information and/or motion information of the user. According to certain embodiments, the processor 250 may determine to output the motion information of the object and/or biometric information and/or motion information of the user to an external electronic device including a display, in response to visually (e.g., text, figure) implementing of the motion information of the object and/or the biometric information and/or motion information of the user.

According to an embodiment, the processor 250 may determine to output the posture information of the object to an external electronic device equipped with a sound output function, in response to implementing of the posture information of the object in an audio form.

According to certain embodiments, in operation 1600, the processor 250 may transmit the feedback information to the determined external electronic device for output thereof.

According to an embodiment, the processor 250 may control a communication module (e.g., the communication module 290 of FIG. 2) to transmit the feedback information to the external electronic device through a network (e.g., the first network 198 and/or the second network 199 of FIG. 1).

FIG. 4 is a diagram illustrating an electronic device according to certain embodiments described in this document and an external electronic device related to the electronic device.

According to certain embodiments, the electronic device 200 may be connected to at least one of a first external electronic device 300, a second external electronic device 400, and a third external electronic device 500. According to an embodiment, the electronic device 200 may control a communication module (e.g., the communication module 290 of FIG. 2) to communicate with at least one of the first external electronic device 300, the second external electronic device 400, and the third external electronic device 500 through a network (e.g., the first network 198 of FIG. 1). According to an embodiment, the first external electronic device 300, the second external electronic device 400, and the third external electronic device 500 may be interconnected through the network.

According to certain embodiments, the electronic device 200 may receive a variety information from the first external electronic device 300, the second external electronic device 400, and/or the third external electronic device 500. According to an embodiment, the electronic device 200 may receive biometric information and/or motion information of a user from the second external device 400, which may be capable of collecting the biometric information (e.g., heart rate and/or calories) and/or motion information (e.g., motion) of the user.

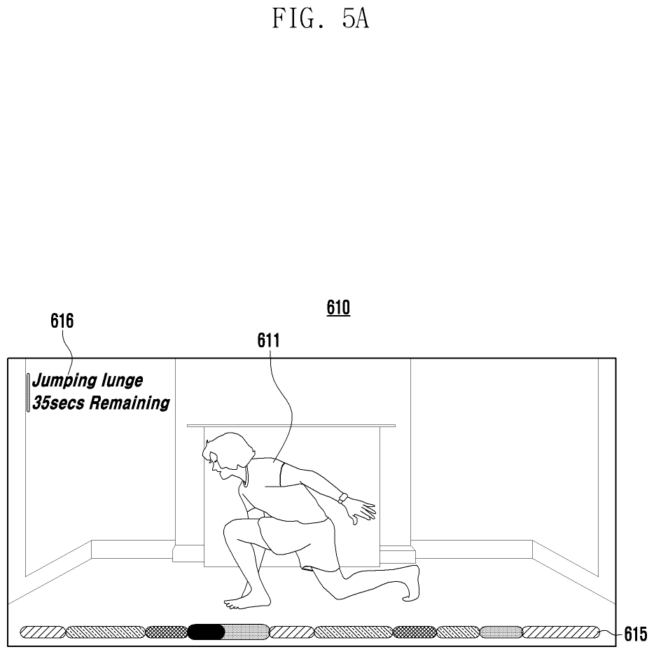

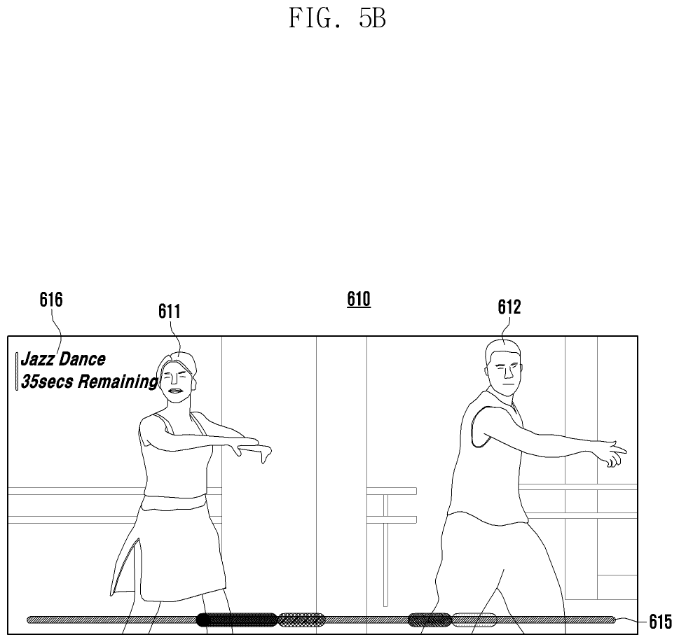

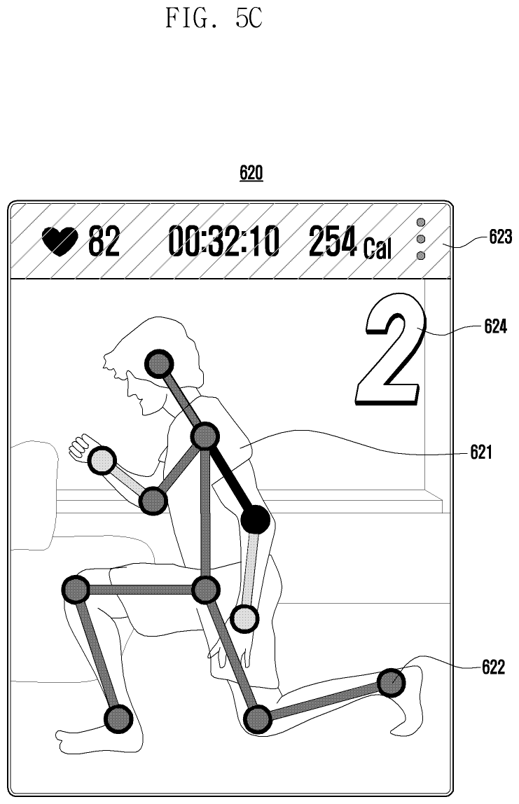

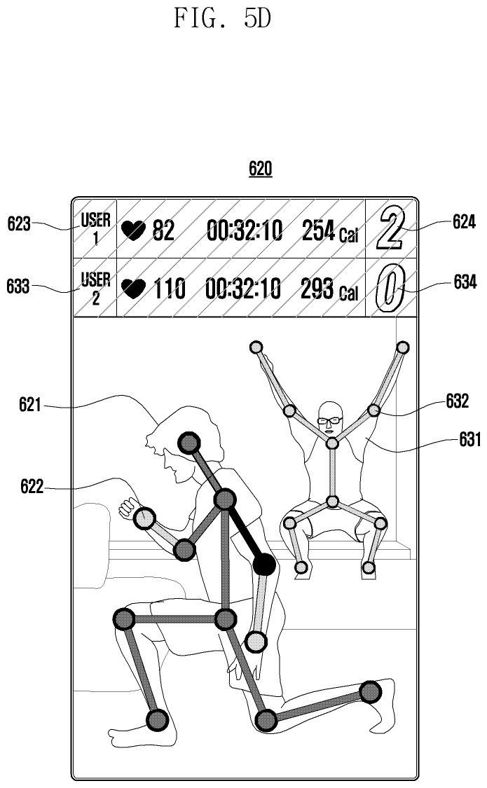

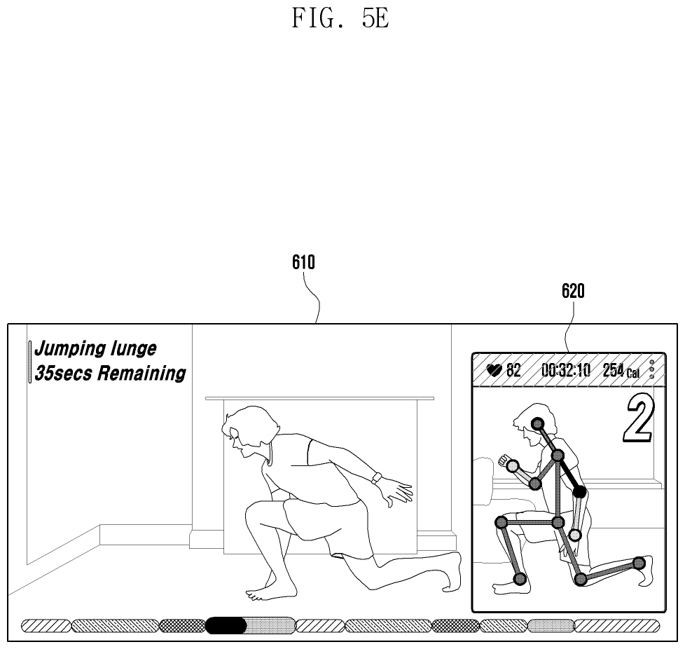

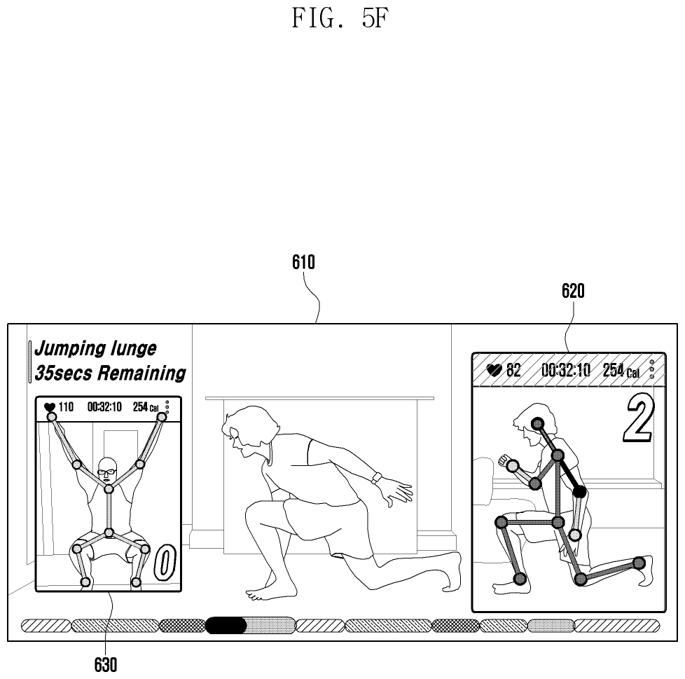

According to certain embodiments, the first external electronic device 300 may be an electronic device (e.g., a TV, a monitor, a projector) including a display. According to certain embodiments, the first external electronic device 300 may include a sound output module (e.g., a speaker). According to certain embodiments, the first external electronic device 300 may display various information received from the electronic device 200 on the display, or output the same to the sound output module. According to an embodiment, the first external electronic device 300 may display a content screen (e.g., a content screen 610 of FIG. 5A) received from the electronic device 200 and/or a user screen (e.g., a user screen 620 of FIG. 5C) on the display. According to an embodiment, the second external electronic device 300 may output the posture information of the object and/or feedback information received from the electronic device 200 to the speaker.

According to certain embodiments, the second external electronic device 400 may be an electronic device (e.g., a wearable device and/or a smart watch) including a function of measuring the biometric information (e.g., heart rate and/or calories) and/or motion information (e.g., motion) of the user. According to an embodiment, the second external electronic device 400 may transmit the measured biometric information and/or motion information of the user to the electronic device 200. According to certain embodiments, the second external electronic device 400 may include a display. According to an embodiment, the second external electronic device 200 may display the measured biometric information and/or motion information of the user on the display. The second external electronic device 400 may display various information received from the electronic device 200 on the display. According to an embodiment, the second external electronic device 400 may display the motion information (e.g., motion section, motion name) of the object received from the electronic device 200 on the display.

According to certain embodiments, the third external electronic device 500 may include a sound output module (e.g., a hearable device, a speaker, and/or an earphone). According to certain embodiments, the third external electronic device 500 may output various information received from the electronic device 200 in the form of audio. According to an embodiment, the third external electronic device 500 may output the posture information and/or feedback information of the object received from the electronic device 200 in the form of audio.

According to certain embodiments, the processor (e.g., the processor 250 of FIG. 2) of the electronic device 200 may control the electronic device 200, the first external electronic device 300, the second external electronic device 400, and/or the third external electronic device 500 to output the various information related to a motion of the object and/or a motion of the user in the content. The processor 250 may determine an electronic device to output information, based on an attribute of the information to be transmitted and/or a function of the electronic device.

Table 1 is a table illustrating functions of an electronic device, which can be performed by the first external electronic device, the electronic device, the second external electronic device, and the third external electronic device, according to an embodiment.

TABLE 1

| | 1^{st} | | | |
|---|---|---|---|---|
| | external | | 2^{nd} | 3^{rd} |
| | electronic | electronic | electronic | electronic |
| Function | device | device | device | device |
| Content screen display | ◯ | ◯ | X | X |
| User screen display | Δ | ◯ | X | X |
| Output of object posture | ◯ | ◯ | X | ◯ |
| information and/or | | | | |
| feedback information in | | | | |
| audio format | | | | |
| Receive transition input | X | ◯ | ◯ | ◯ |
| to another action | | | | |
| Generation user's | X | ◯ | X | X |
| posture information | | | | |
| Measurement of user's | X | X | ◯ | X |
| biometric information | | | | |
| Measurement user's | X | X | ◯ | X |
| motion information | | | | |
| Display of user's | ◯ | ◯ | ◯ | X |
| biometric information | | | | |
| and/or motion | | | | |
| information | | | | |

Referring to Table 1, the meaning is as follows: O: Provides the corresponding function, X: Does not provide the corresponding function, A: Can provide depending on the conditions. According to an embodiment, the processor 250 may control the first external electronic device 300 and/or the electronic device 200 to display a content screen (e.g., the content screen 610 of FIG. 5A).

According to an embodiment, the processor 250 may control the first external electronic device 300 and/or the electronic device 200 to display a user screen (e.g., the user screen 620 of FIG. 5C). According to an embodiment, the processor 250 may configure the user screen 620 by obtaining an image including the user captured by a camera 270 included in the electronic device 200 and/or a camera included in the external electronic device.

According to an embodiment, the processor 250 may control one of the first external electronic device 300, the electronic device 200, and the third external electronic device 500 to output the posture information and/or feedback information of the object in the content in a form of audio. According to an embodiment, the processor 250 may control one of the devices to output one of the information, based on one of a user configuration, an attribute of information, and a specified condition in response to the fact that two or more devices are connected to the electronic device 200. For example, the processor 250 may control one of the first external electronic device 300 and/or the electronic device 200 to output the posture information of the object and may control the third electronic device 500 to output the feedback information.

According to an embodiment, the processor 250 may receive a motion change input of the user for switching an action being output from the content to another action within the content from one of the electronic device 200, the second external electronic device 400, and the third external electronic device 500. According to an embodiment, the processor 250 may receive an input from all connected electronic devices and/or may receive an input from a preset electronic device among the connected electronic devices, in response to the fact that two or more devices are connected to the electronic device 200.

According to an embodiment, the processor 250 may generate posture information of the user by using the image including the user. The processor 250 may analyze the posture of the user included in the image obtained through the camera, and generate posture information of the user based on the analyzed posture.

According to an embodiment, the processor 250 may control to obtain the biometric information (e.g., heart rate and/or calories) information and/or motion information (e.g., number of operations and/or operation time) of the user through the second external electronic device 400.

According to an embodiment, the processor 250 may control at least one of the first external electronic device 300, the electronic device 200, and the second external electronic device 400 such that at least one of the first external electronic device 300, the electronic device 200, and the second external electronic device 400 displays the biometric information and/or motion information of the user. According to an embodiment, the processor 250 may control the first external electronic device 300 and/or the electronic device 200 to display the biometric information and/or motion information of the user on the first external electronic device 300 and/or the electronic device 200 in the form including the user's screen 620, and/or may control the second external electronic device 400 to display the biometric information and/or motion information of the user on the second external electronic device 400 in the form of text or an icon.

The processor 250 may control the electronic device 200, the first external electronic device 300, the second external electronic device 400, and the third external electronic device 500 to generate, obtain, and/or output various information related to a motion of the object and/or a motion of the user in the content, in response to various situations related to the type, number, and operation time of the electronic devices available to the user.

According to an embodiment, the types of electronic devices may be classified according to whether a display is included, a size of the display, whether a camera is included, whether a sound output module is included, whether a biometric sensor is included, whether a motion detection sensor is included, and/or whether an input module is included. According to an embodiment, the processor 250 may control a device including a display of a specified size or larger to display the content screen and/or the user screen and to display the user's biometric information and/or motion information in a form that includes the user's screen, and may control the device including the display smaller than the specified size to display the user's biometric information and/or motion information in the form of text and/or icons on the device including the display smaller than the specified size. According to an embodiment, the processor 250 may configure a user screen by obtaining an image including the user from a device including a camera. According to an embodiment, the processor 250 may control a device including a sound output module to output the posture information and/or feedback information of the object in the form of audio to the device including the sound output module. According to an embodiment, the processor 250 may control a device including a biosensor and/or a motion sensor to obtain the user's biometric information and/or motion information from the device including the biometric sensor and/or the motion sensor. According to an embodiment, the processor 250 may control a device including various types of input modules to obtain a user's motion change input from the devices including various types of input modules. According to an embodiment, the first external electronic device 300 may include a display having a predetermined size or larger, a sound output module, and/or a camera. In addition, the electronic device 200 may include a display having a predetermined size or larger, a sound output module, an input module, and/or a camera. In addition, the second external electronic device 400 may include a display smaller than a predetermined size, a biometric sensor, a motion detection sensor, and/or an input module. Furthermore, the third external electronic device 500 may include a sound output module and/or an input module.

According to an embodiment, in response to the fact that there are a plurality of devices capable of performing the same function among the electronic devices available to the user, the processor 250 may control the plurality of devices to perform the function in all of the devices and/or to perform the function in some devices. For example, when there are a plurality of devices including a display, the processor 250 may control the plurality of devices including the display to display the user's biometric information and/or motion information on all devices including the display, to display the user's biometric information on some of the devices including the display and display the user's motion information on some of the devices including the display, or to display the user's biometric information and/or motion information on some of the devices including the display. For another example, when there are a plurality of electronic devices including displays having a size greater than or equal to a specified size, the processor 250 may control the plurality of devices including the display of the specified size or larger to display a content screen and/or a user screen on all the devices including the display of specified sizes or larger, or to display a content screen on some of the electronic devices including the display having a size greater than or equal to the specified size and display a user screen on some of the electronic devices including the display having a size greater than or equal to the specified size. As another example, when there are a plurality of devices including a sound output module, the processor 250 may control the plurality of devices including the sound output module to output posture information and/or feedback information of the object in audio form to all the devices including the sound output module, or to output the posture information of the object to some of the devices including the sound output module and output feedback information to some of the devices including the sound output module.

According to an embodiment, the processor 250 may output the posture information and/or feedback information of the object in the form of audio to the device including the sound output module, based on the execution time of a motion. For example, in response to the fact that the execution time of the motion corresponds to the daytime, the processor 250 may control the device including the sound output module to output posture information and/or feedback information of the object in an audio format from a device in which the sound output module is in the form of a speaker. For another example, in response to the fact that the execution time of the motion corresponds to the night time, the processor 250 may control the device including the sound output module to output the posture information and/or feedback information of the object in the form of audio from the device in which the sound output module is in the form of an earphone. According to an embodiment, in response to the fact that the electronic devices available to the user are the electronic device 200, the first external electronic device 300, and the second external electronic device 400, the processor 250 may control the electronic device 200 to display the user screen 620, to generate the user's posture information, and to display the user's biometric information and/or motion information, may control the first external electronic device 300 to display the content screen 610 and to output the posture information and/or feedback information of the object in the form of audio, and may control the second external electronic device 400 to obtain a user's motion change input and to obtain the user's biometric information and/or motion information.

According to an embodiment, in response to the fact that the electronic devices available to the user are the electronic device 200, the first external electronic device 300, and the second external electronic device 400, the processor 250 may control the electronic device 200 to display the content screen 610 and the user screen 620, to generate the user's posture information, to output the posture information and/or feedback information of the object in the form of audio, and may control the second external electronic device 400 to obtain the user's motion change input and to obtain the user's biometric information and/or motion information.

According to certain embodiments of the disclosure, the processor 250 may select an electronic device to output feedback information among various electronic devices connected (e.g., the first external electronic device 300, the second external electronic device 400, and/or the third external electronic device 500) to the electronic device 200 and may transmit the feedback information to the selected electronic device. The processor 250 may differently select the electronic device to output the feedback information according to a time to output the feedback information.

According to an embodiment, in response to the fact that the devices available to the user are the electronic device 200, the first external electronic device 300, the second external electronic device 400, and the third external electronic device 500 and the execution time of the action of feeding back the user's motion on the action of the object in the content is a specific time (e.g., night time), the processor 250 may control the electronic device 200 to display the user screen 620, to generate the user's posture information, and to display the user's biometric information and/or motion information, may control the first external electronic device 300 to display the content screen 610, may control the second external electronic device 400 to obtain the user's motion change input and to obtain the biometric information and/or motion information, and may control the third external electronic device 500 to output the posture information and/or feedback information of the object in the audio form.

According to an embodiment, in response to the fact that the devices available to the user are the electronic device 200, the first external electronic device 300, the second external electronic device 400, and the third external electronic device 500, and the execution time of the motion of feeding back the user's motion with respect to the motion of the object in the content is a specific time (e.g., daytime), the processor 250 may control the electronic device 200 to display the user screen 620, to generate the user's posture information, and to display the user's biometric information and/or motion information, may control the first external electronic device 300 to display the content screen 610 and to output the posture information and/or feedback information of the object in the form of audio, may control the second external electronic device 400 to obtain the user's biometric information and/or motion information, and may control the third external electronic device 500 to obtain the user's motion change input.

According to an embodiment, in response to the fact that the devices available to the user are the first external electronic device 300 and the second external electronic device 400, the processor 250 may control the first external electronic device 300 to display the content screen 610, to output the posture information and/or feedback information of the object in the audio format, and to display biometric information and/or motion information of the user, and may control the second external electronic device 400 to obtain the user's motion change input and to obtain the user's biometric information and/or motion information.

According to an embodiment, in response to the fact that the devices available to the user are the electronic device 200 and the second external electronic device 400, the processor 250 may control the electronic device 200 to display the content screen 610 and the user screen 620, to generate the user's posture information, and to display the user's biometric information and/or motion information, may control the second external electronic device 400 to obtain a motion change input of the user and to output the posture information and/or feedback information of the object in the form of audio.

According to certain embodiments, in response to the fact that there are multiple electronic devices and/or multiple external electronic devices belonging to multiple users, the processor 250 may determine an electronic device to output information based on one of a property of information to be output, a function of the electronic device, the number of users, and a relationship between users and an external electronic device. For example, in response to the fact that there are a plurality of users, the processor 250 may control a device associated with each user to generate each user's posture information and/or feedback information, to obtain each user's biometric information and/or motion information, and to output the biometric information and/or motion information of the user from the device related to each user.

Table 2 is a table illustrating functions of the electronic devices, which can be performed by the first external electronic device, the electronic device of first user, the second external electronic device of the first user, the third external electronic device of the first user, the electronic device of the second user, the second external electronic device of the second user, and the third external electronic device of the second user according to an embodiment.

TABLE 2

| | | | 1st user's | 1st user's | | 2^{nd} user's | 2^{nd} user's |
|---|---|---|---|---|---|---|---|
| | 1^{st} | | 2^{nd} | 3^{rd} | | 2^{nd} | 3^{rd} |
| | external | 1^{st} user's | external | external | 2^{nd} user's | external | external |
| | electronic | electronic | electronic | electronic | electronic | electronic | electronic |
| Function | device | device | device | device | device | device | device |
| Display of | ◯ | ◯ | X | X | ◯ | X | X |
| content | | | | | | | |
| screen | | | | | | | |
| Display of | Δ | ◯ | X | X | ◯ | X | X |
| user screen | | | | | | | |
| Output of | ◯ | ◯ | X | ◯ | ◯ | X | ◯ |
| object | | | | | | | |
| posture | | | | | | | |
| information | | | | | | | |
| and/or | | | | | | | |
| feedback | | | | | | | |
| information | | | | | | | |
| in audio | | | | | | | |
| format | | | | | | | |
| Reception of | X | ◯ | ◯ | ◯ | ◯ | ◯ | ◯ |
| transition | | | | | | | |
| input to | | | | | | | |
| another | | | | | | | |
| motion | | | | | | | |
| Generation | X | ◯ | X | X | ◯ | X | X |
| of user's | | | | | | | |
| posture | | | | | | | |
| information | | | | | | | |
| Measurement | X | X | ◯ | X | X | ◯ | X |
| of user's | | | | | | | |
| biometric | | | | | | | |
| information | | | | | | | |
| Measurement | X | X | ◯ | X | X | ◯ | X |
| of user's | | | | | | | |
| motion | | | | | | | |
| information | | | | | | | |
| Display of | ◯ | ◯ | ◯ | X | ◯ | ◯ | X |
| user's | | | | | | | |
| biometric | | | | | | | |
| information | | | | | | | |
| and/or | | | | | | | |
| motion | | | | | | | |
| information | | | | | | | |

Referring to Table 2, that the meaning is as follows: O: Provides the corresponding function, X: Does not provide the corresponding function, A: Can provide depending on the conditions.

According to an embodiment, the processor 250 may control one of the first external electronic device 300, the electronic device of the first user, and the electronic device of the second user to display the content screen 610.

According to certain embodiments, the processor 250 may control one of the first external electronic device 300, the electronic device of the first user, and the electronic device of the second user to display the user screen 620. According to an embodiment, the processor 250 may control the first external electronic device 300 to display a user screen (e.g., the user screen 620 of FIG. 5D) including the images of the first user and/or the second user obtained from the camera provided in the electronic device of the first user and/or the camera provided in the electronic device of the second user. According to another embodiment, the processor 250 may control the first external electronic device 300 to display at least one of a first user screen (e.g., the user screen 620 of FIG. 5F) including an image of the first user obtained from the electronic device of the first user and a second user screen (e.g., the user screen 630 of FIG. 5F) including an image of the second user obtained from the electronic device of the second user to display at all.

According to an embodiment, the processor 250 may control one of the first external electronic device 300, the electronic device of the first user, the electronic device of the second user, the third electronic device of the first user, and the third electronic device of the second user to output the posture information of the object and/or feedback information according to the comparison of the posture information of the object in the content and the posture information of the user in the content in the form of audio. According to an embodiment, the processor 250 may control one of the first external electronic device 300, the electronic device of the first user, and the electronic device of the second user to output the posture information of the object in the form of audio. According to an embodiment, the processor 250 may control the third electronic device of the first user to output the first feedback information on the first user and the third electronic device of the second user to output the second feedback information on the second user in the form of audio. For example, the processor 250 may classify the device (e.g., the first external electronic device 300 and/or the electronic device 200) used by the first user and the second user simultaneously as a common device and control the devices to output the posture information of the object in the form of audio, and may classify the devices (e.g., the electronic device, the third external electronic device of the first user, and/or the electronic device, third external electronic device of the second user) used by the first user and the second user as personal devices and control each device such that the first feedback information is output from the personal electronic device of the first user (e.g., the electronic device of the first user and/or the third external electronic device of the first user) and the second feedback information is output from the personal electronic device of the second user (e.g., the electronic device of the second user and/or the third external electronic device of the second user) in the form of audio. For example, a case in which the electronic device 200 is classified as a public device may include a case in which the electronic device 200 displays a content screen, and a case in which the electronic device 200 is classified as a personal device may include a case in which the first external electronic device 300 displays a content screen.

According to an embodiment, the processor 250 may receive a motion change input for switching a motion of a user being output from the content to another motion in the content from one of the electronic device of the first user, the electronic device of the second user, the second external electronic device of the first user, the second external electronic device of the second user, the third external electronic device of the first user, and the third external electronic device of the second user. According to an embodiment, the input may be received from all the devices and/or the input may be received from a preset device among the devices. The reset device capable of receiving the input may be configured by the user.

According to an embodiment, the processor 250 may control the electronic device of the first user and/or the electronic device of the second user to generate the posture information of the first user and/or second user. According to an embodiment, in response to the fact that the electronic device of the first user and the electronic device of the second user are used at the same time, the processor 250 may control the electronic device of the first user and/or the electronic device of the second user such that the electronic device of the first user generates the posture information of the first user included in the screen captured by the camera provided in the electronic device of the first user, and the electronic device of the second user generates the posture information of the second user included in the screen captured by the camera provided in the electronic device of the second user. According to another embodiment, in response to the fact that the electronic device of the first user is used, the processor 250 may control the electronic device of the first user to generate the posture information of the first user and/or second user included in the screen captured by the camera 270 included in the electronic device of the first user.

According to an embodiment, the processor 250 may receive the biometric information and/or motion information of the first user measured by the second external electronic device of the first user and/or the biometric information and/or motion information of the second user measured by the second external electronic device of the second user.

According to an embodiment, the processor 250 may control at least one of the first external electronic device 300, the electronic device of the first user, and the electronic device of the second user to display biometric information (e.g., heart rate and/or or calories) and/or motion information (e.g., number of motions and/or duration of motion) of the first user and/or the second user. In addition, the processor 250 may control the second external electronic device of the first user to display the biometric information and/or motion information of the first user, and may control the second external electronic device of the second user to display the biometric information and/or motion information of the second user. For example, the processor 250 may control one of the first external electronic device 300, the electronic device of the first user, and the electronic device of the second user to display the biometric information and/or motion information of the first user and/or second user on one of the devices in a form in which the biometric information and/or motion information of the first user and/or second user are included in the first user screen 620 and/or the second user screen 630, or may control the second external electronic device of the first user and/or the second external electronic device of the second user to display in the form of text or icons on the second external electronic device of the first user and/or the second external electronic device of the second user.

According to certain embodiments, the processor 250 may control to display the content screen (e.g., the content screen 610 of FIG. 5A) and/or the user screen (e.g., the user screen 620 of FIG. 5C) on the display (e.g., the display 220 of FIG. 2) of the electronic device (e.g., the electronic device 200 of FIG. 2) or to display on an external electronic device (e.g., the second external device 300 of FIG. 4) including a display.

FIG. 5A is a diagram illustrating an example of the content screen 610, according to certain embodiments described herein.

According to certain embodiments, the processor 250 may display the content screen 610 including image information of the content stored in the memory (e.g., the memory 280 of FIG. 2). According to an embodiment, the content may include various images (e.g., exercise images for home training) obtained from an external media source.

According to certain embodiments, the content screen 610 may include an object 611 that performs a motion in the content (e.g., a trainer in a shared online video, performing an exercise). According to an embodiment, the processor 250 may distinguish the background area and the object 611 in the content. The processor 250 may analyze the image information of the object 611 to generate motion information of the object, including segmentation of the motion performed by the object and the name of the motion, and/or the posture information of the object including relative position information of each part of the body of the object performing the motion. In addition, the processor 250 may generate feedback information by comparing the posture information of the object with the posture information of the user.

The processor 250 may process the motion information of the object in various forms to display the motion information on the content screen 610. For example, the processor 250 may display the motion information of the object in the form of text 616 and/or block bar 615 on the content screen 610.

According to certain embodiments, the processor 250 may generate section information by dividing an operation section of the content, based on the motion information of the object. For example, the processor 250 may divide the section in which the object 611 performs a first operation (e.g., a lunge motion) as a first interval, and divide the section in which the object 611 performs a second operation (e.g., a squat motion) as a second section.

The processor 250 may generate information on the start position, end position, and length of each section in the content as section information of each section. According to an embodiment, the processor 250 may display section information in the form of a block bar 615 on the content screen 610. The processor 250 may differently display section information of the first section (e.g., a section in which a lunge motion is performed) and section information of the second section (e.g., a section in which a squat motion is performed). For example, the processor 250 may display the block bar 615 by classifying colors for each section of the content. In addition, the processor 250 may display the currently ongoing first operation section in the form of a thicker block bar (e.g., the fourth block from the left among the block bars 615 of FIG. 5A) compared to other block bars.

According to an embodiment, the processor 250 may display, on the content screen 610, information indicating the degree of progress of the motion, based on the motion information of the object. For example, the processor 250 may display the progress position of the current motion on the block bar 615 in the first operation section currently in progress. As another example, in the first motion section, the processor 250 may display the remaining motion time as a text 616 on the content screen 610.

According to an embodiment, the processor 250 may display the content by variously utilizing the section information according to the type of content (e.g., difficulty and type of exercise within the content) and type of motion (e.g., difficulty and type of motion). For example, in the case of a first content type (e.g., beginner exercise with low difficulty), the processor 250 may display the content by using the section information of the motion, such as displaying a first motion (e.g., Jumping Jack) in the first section for 30 seconds, displaying a second motion (e.g., Walk into Plank) in the second section for 30 seconds, then displaying the first motion and the second motion again in succession, and stopping the display of the content so that the user can rest for 30 seconds thereafter.

In another embodiment, in the case of the second content type (e.g., expert exercise with high difficulty), the processor 250 may display the content by using the section information of the motion, such as displaying a first motion (e.g., Steam Engines) for 60 seconds in the first section, displaying a second motion (e.g., Squats) in the second section for 60 seconds, displaying a third exercise (e.g., Burpees) in the third section for 60 seconds, and then stopping the display of the content so that the user can rest for 20 seconds.

According to an embodiment, the processor 250 may display the content as a detailed division motion including a series of steps within one section according to the type of the motion. As an embodiment, in the case of a third content type (e.g., yoga), the processor 250 may display the content by using the section information of the motion, such as dividing and displaying the first motion (e.g., Side Rotation) into a first detailed division motion (e.g., To_left) and a second detailed division motion (e.g., To_right) for 60 seconds in the first section, displaying a second motion (e.g., Cat cow) in the second section for 60 seconds, and dividing and displaying the third motion (e.g., Balancing Table) into a third detailed division motion (e.g., To_left) and a fourth detailed division motion (e.g., To_right) in the third section for 90 seconds.

According to an embodiment, the processor 250 may display the content screen 610 according to the type of motion (e.g., a motion performed while sitting). For example, in the motion of performing the upper body, the content may be displayed for the upper body part of the object, and the posture information of the object may be provided for the motion of the upper body of the object.

According to an embodiment, the processor 250 may generate the number of repetitions of a motion as section information according to the type of content and/or the type of motion. For example, in the case of the fourth content (e.g., dancing), information of repeating the first motion (e.g., step) twice in the first section, repeating the second motion (e.g., elbow rhythm) three times in the second section, and repeating the third motion (e.g., backstep) twice in the third section may be generated as section information, and the fourth content may be displayed according to the section information.

According to certain embodiments, the processor 250 may determine the motion name, based on the motion information of the object. According to an embodiment, in response to the object 611 performing the first action, the processor 250 may match a general motion name (e.g., jumping, lunge, wide squat, burpee test, crunch, dance) of the first motion. The processor 250 may display the motion name as a text 616 on the content screen 610.

FIG. 5B is a diagram illustrating an example of a content screen, according to certain embodiments described in this document. The example of FIG. 5B is a diagram illustrating an example of the content screen 610 for content that includes an exercise-related motion in a partial section.

According to certain embodiments, the processor 250 may display the content screen 610 including image information of the content stored in the memory 280. According to an embodiment, the content may include various images (e.g., a movie including an exercise section, a drama) obtained from an external media source.

According to certain embodiments, the content screen 610 may include a first object 611 and/or a second object 612 performing motions within the content (e.g., a jazz dance motion). According to an embodiment, the processor 250 may distinguish the background area from the first object 611 and/or the second object 612 in the content. The processor 250 may analyze the image information of the first object 611 and/or the second object 612 to generate motion information of each of the first object 611 and/or the second object 612 and/or the posture information of each of the first object 611 and/or the second object 612.

The processor 250 may process the motion information of the object in various forms and display the same on the content screen 610. For example, the processor 250 may display the motion information in the form of a text 616 and/or a block bar 615 on the content screen 610.

According to certain embodiments, the processor 250 may generate section information by dividing an operation section of the content based on the motion information of the object. According to an embodiment, the processor 250 may classify a section in which a motion (e.g., exercise, dancing, cooking) related to a field in which content is designated is performed based on the motion information of the object. For example, the processor 250 may classify a section in which the first object 611 and/or the second object 612 performs a first motion as a first section, and classify a section in which the first object 611 and/or the second object 612 performs the second motion as a second section. The processor 250 may generate information on a start position, an end position, and a length of each section in the content as section information of each section. According to an embodiment, the processor 250 may display the section information in the form of a block bar 615 on the content screen 610. The processor 250 may differently display the section information of the first section, section information of the second section, and section information of a section that does not include a motion. For example, in response to content that includes some sections of the action (e.g., exercise, dancing, cooking) in a specified field, the processor 250 may display the block bar 615 by classifying colors for each section with respect to the first section and/or the second section performing a motion in a designated field, and may not display the block bar 615 for a section that does not include an operation in a designated field. Furthermore, the processor 250 may display the currently ongoing first operation section in the form of a block bar thicker than other block bars.

FIG. 5C is a diagram illustrating an example of the user screen 620 including a user, according to certain embodiments described herein.

According to certain embodiments, the processor 250 may obtain an image including the user 621 through the camera.

According to an embodiment, the camera through which the processor 250 may obtain an image including the user may include a camera (e.g., the camera 270 of FIG. 2) included in the electronic device 200.

According to an embodiment, the camera through which the processor 250 obtains the image including the user may include a camera (e.g., a webcam, a camcorder, or a wireless camera) provided in an external electronic device, and the camera provided in the external electronic device may be connected to the electronic device 200 by wire and/or wirelessly.

According to certain embodiments, the processor 250 may distinguish the background area from the user 621 in the user screen 620. The processor 250 may analyze the image information of the user 621 to generate the posture information of the user.

According to certain embodiments, the processor 250 may compare the posture information of the object and the posture information of the user in the content, and may generate feedback information, based on the comparison result. According to an embodiment, the processor 250 may check the similarity between the posture information of the object and the posture information of the user based on the feedback information, and may divide the posture of the user 621 into at least two or more regions based on the similarity. For example, the processor 250 may divide the posture of the user into a matching region (e.g., a region in which the degree of similarity is equal to or greater than a first value), a similar region (e.g., a region in which the similarity is less than the first value and greater than or equal to a second value), and/or dissimilar region (e.g., a region in which the degree of similarity is less than the second value), based on the similarity. The processor 250 may visualize the divided regions and display the visualized regions on the display. The processor 250 may display the visualized regions by overlaying the visualized regions on the user screen 620.

The processor 250 may display the feedback information on the user screen 620 through various visualized forms. According to an embodiment, the processor 250 may display the feedback information as a guide figure 622 visualized in the form of an abstract figure, using nodes and lines. For example, the processor 250 may display the guide figure 622 by dividing the guide figure into nodes and/or lines. The nodes may represent at least rotational portions of the body (e.g., joints) and/or terminal regions of the body (e.g., the head). The lines may represent at least generally inflexible portions thereof that connect the nodes, such as the limbs. The processor 250 may display a node to correspond to a part (e.g., a joint such as wrist, elbow, head, pelvis, and popliteal muscle) of a body of the user 621, and may display a line connecting the nodes. According to an embodiment, the processor 250 may differently display the colors of lines connecting the nodes in response to information that divides the posture of the user 621 into the matching region, the similar region, and the dissimilar region based on the similarity (e.g., the matching region with a first color line, the similar region with a second color line, the dissimilar region with a third color line). Accordingly, the user can intuitively identify the body part different from the posture of the object in the content.

According to certain embodiments, the processor 250 may obtain the biometric information (e.g., heart rate and/or calories) and motion information (e.g., movement) of the user received by the communication module 290 from an external device (e.g., the second external device 400). The processor 250 may display the biometric information 623 and/or motion information 624 of the user on the user screen 620. For example, the processor 250 may display the biometric information 623 including the heart rate and/or calories of the user on the user screen 620, and may display the motion information (e.g., the number of motions) 624 of the user on the user screen.

FIG. 5D is a diagram illustrating an example of the user screen 620 including a plurality of users, according to certain embodiments described in this document.

According to certain embodiments, the processor 250 may obtain an image including a first user 621 and/or a second user 631 through a camera.

According to an embodiment, the camera through which the processor 250 obtains the image including the user may include a camera (e.g., the camera 270 of FIG. 2) included in the electronic device 200.

According to an embodiment, the camera through which the processor 250 obtains the image including the user may include a camera (e.g., a webcam, a camcorder, a wireless camera) provided in an external electronic device, and the camera provided in the external electronic device may be connected to the electronic device 200 by wire and/or wirelessly.

According to certain embodiments, the processor 250 may distinguish the background area from the first user 621 and/or the second user 631 in the user screen 620. The processor 250 may analyze image information of the first user 621 and/or the second user 631 to generate posture information of the first user and/or the second user.

According to certain embodiments, the processor 250 may compare the posture information of each of the first user 621 and/or the second user 631 with the posture information of the object in the content, and may generate first feedback information of the first user 621 and/or second feedback information of the second user 631, based on the comparison result. According to an embodiment, the processor 250 may check the similarity between the posture information of the object and the posture information of the first user 621 and/or the second user 631, based on the first feedback information and/or the second feedback information, and may divide the postures of the first user 621 and/or the second user 631 into at least two regions, based on each similarity. For example, the processor 250 may divide the posture of the first user 621 and/or the second user 631 into a matching region (e.g., a region in which the similarity is equal to or greater that a first value), a similar region (e.g., a region in which the similarity is less than a first value and greater than or equal to a second value), and/or a dissimilar region (e.g., a region in which the similarity is less than the second value) based on the similarity. The processor 250 may visualize the divided regions and may display the visualized regions on the display. The processor 250 may display the visualized regions on the user screen 620 in a manner of overlaying on the image of the first user 621 and/or the image of the second user 631. The processor 250 may display first feedback information and/or second feedback information on the user screen 620 through various visualized forms. According to an embodiment, the processor 250 may display the first feedback information and/or the second feedback information as a first guide figure 622 and/or a second guide figure 632 visualized in a figure form, respectively.

According to certain embodiments, the processor 250 may obtain biometric information (heart rate and/or calories) and/or motion information of the first user 621 and/or the second user 631 received by the communication module 290 from the first external device (e.g., the second external device 400) and/or the second external device (e.g., the second external device 400). The processor 250 may display the biometric information 623 and/or the motion information 624 of the first user and/or the biometric information 633 and/or the motion information 634 of the second user on the user screen 620. For example, the processor 250 may display the biometric information 623 of the first user including the heart rate and/or calories of the first user and/or the biometric information 633 of the second user including of the heart rate and/or calories of the second user on the user screen 620, and may display the motion information (e.g., the number of motions) 624 of the first user and/or the motion information (e.g., the number of motions) 634 of the second user on the user screen.

FIGS. 5E and 5F are diagrams illustrating examples of displaying content screens and user screens, according to certain embodiments.

According to certain embodiments, the processor 250 may control the content screen 610 and/or the user screen 620 to be displayed on the display 220 of the electronic device 200, or may control to be displayed on an external electronic device including a display (e.g., the second external device 300 of FIG. 4). FIG. 5E is a diagram illustrating an example in which the processor 250 displays the content screen 610 and the user screen 620 on the display 220 of the electronic device 200, and/or the display of the external electronic device 300, in response to the fact that there is one user screen 620 (e.g., when there is one camera for photographing the user). The processor 250 may control the content screen 610 and the user screen 620 to be displayed on one display. According to an embodiment, the properties (e.g., size, ratio, location) of the content screen 610 and/or the user screen 620 may be configured using various methods. For example, the processor 250 may determine the properties of the content screen 610 and/or the user screen 620, based on the properties of the display 220 (e.g., the resolution of the display 220, the aspect ratio of the display 220). As another example, the processor 250 may configured the properties of the content screen 610 and/or the user screen 620, based on the user input for configuring the properties of the content screen 610 and/or the user screen 620.

FIG. 5F is a diagram illustrating an example of a screen configuration displayed on the display in which the processor 250 displays the content screen 610, the first user screen 620, and/or the second user screen 630 on the display 220 of the electronic device 200 and/or the external electronic device 300, in response to the fact that there is a first user screen 620 including the first user, and/or a second user screen 630 including the second user (e.g., when there are a plurality of electronic devices including a camera for photographing the user).

The processor 250 may control the content screen 610, the first user screen 620, and/or the second user screen 630 to be displayed on one display. According to an embodiment, the properties (e.g., size, ratio, and location) of the content screen 610, the first user screen 620, and/or the second user screen 630 may be set by various methods. For example, the processor 250 may determine the properties of the content screen 610, the first user screen 620, and/or the second user screen 630, based on the properties of the display 220 (e.g., the resolution of the display 220, the aspect ratio of the display 220). As another example, the processor 250 may configured the properties of the content screen 610, the first user screen 620, and/or the second user screen 630, based on a user input for configuring the properties of the content screen 610, the first user screen 620 and/or the second user screen 630.

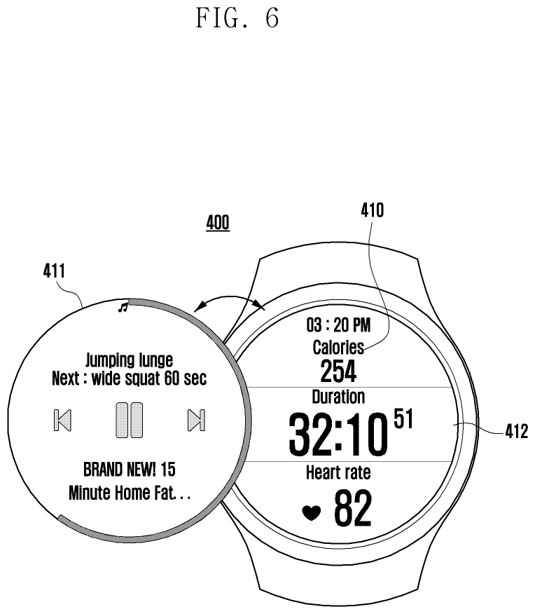

FIG. 6 is a diagram illustrating an example of a second external electronic device 400 connected to an electronic device 200, according to certain embodiments of the disclosure. According to certain embodiments, the second external electronic device 400 may include a display 410 that displays various information on its front surface.

According to certain embodiments, the second external electronic device 400 may display a first screen 411 and/or a second screen 412 on the display 410. According to an embodiment, while displaying the first screen 411, the second external electronic device 400 may switch to display the second screen 412 in response to a user input (e.g., a swipe action) requesting to switch from the first screen 411 to the second screen 412.

According to certain embodiments, the second external electronic device 400 may display information received from the electronic device 200 on the first screen 411. According to an embodiment, various formats (e.g., text, icon) of motion information (e.g., motion name, section information) of an object received from an electronic device (e.g., the electronic device 200 of FIG. 2) may be displayed.

According to certain embodiments, the second external electronic device 400 may display various formats (e.g., text, icon) of the biometric information (e.g., heart rate and/or calories) and/or motion information (e.g., number of operations, operation time) of the user measured by the second external electronic device 200 on the second screen 412.

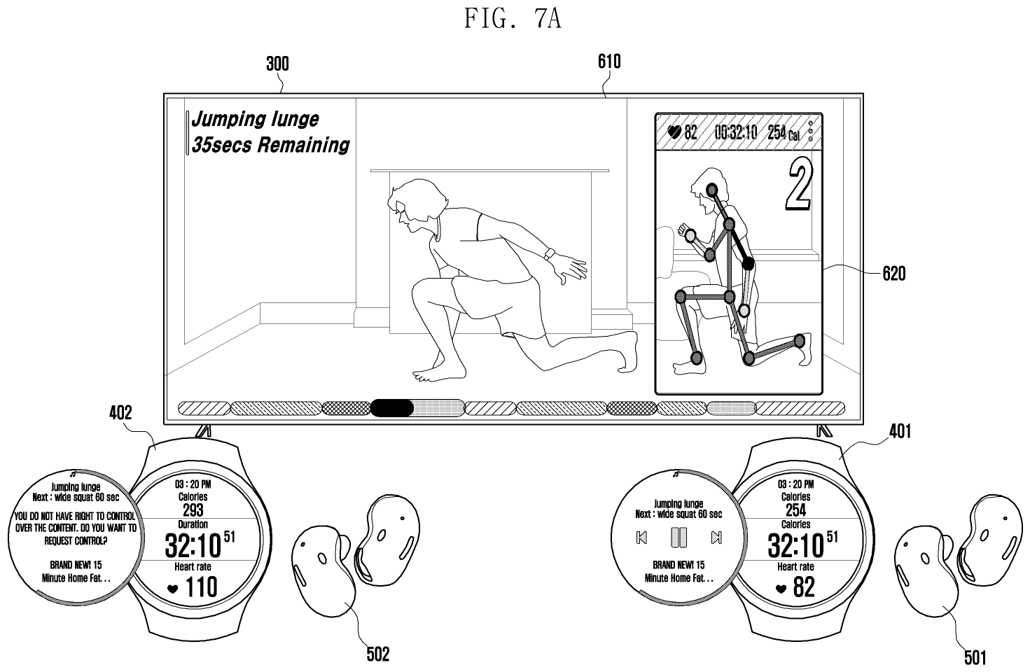

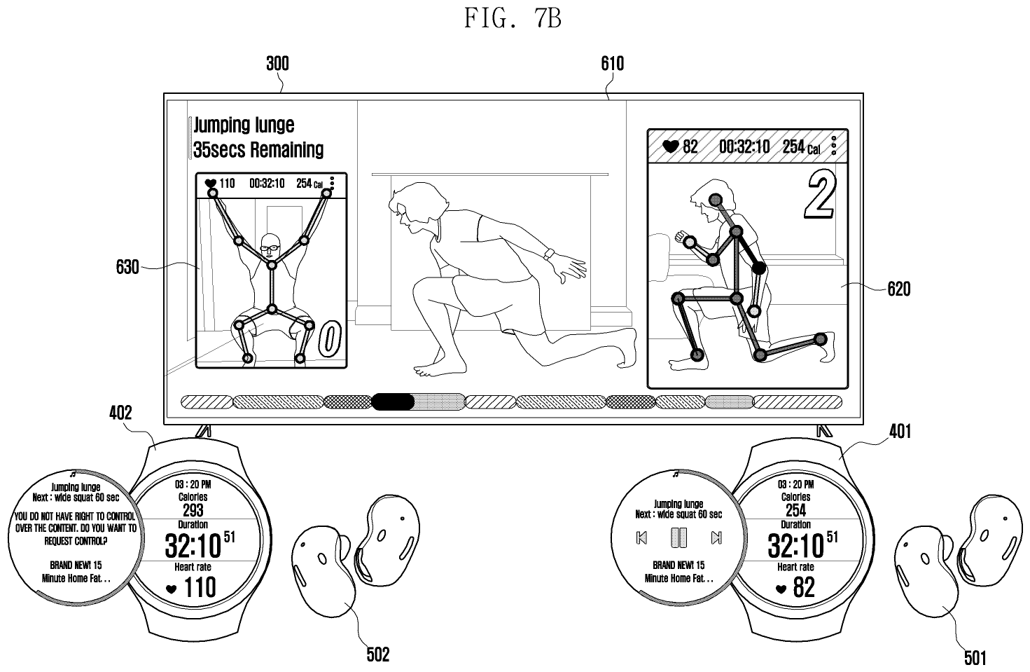

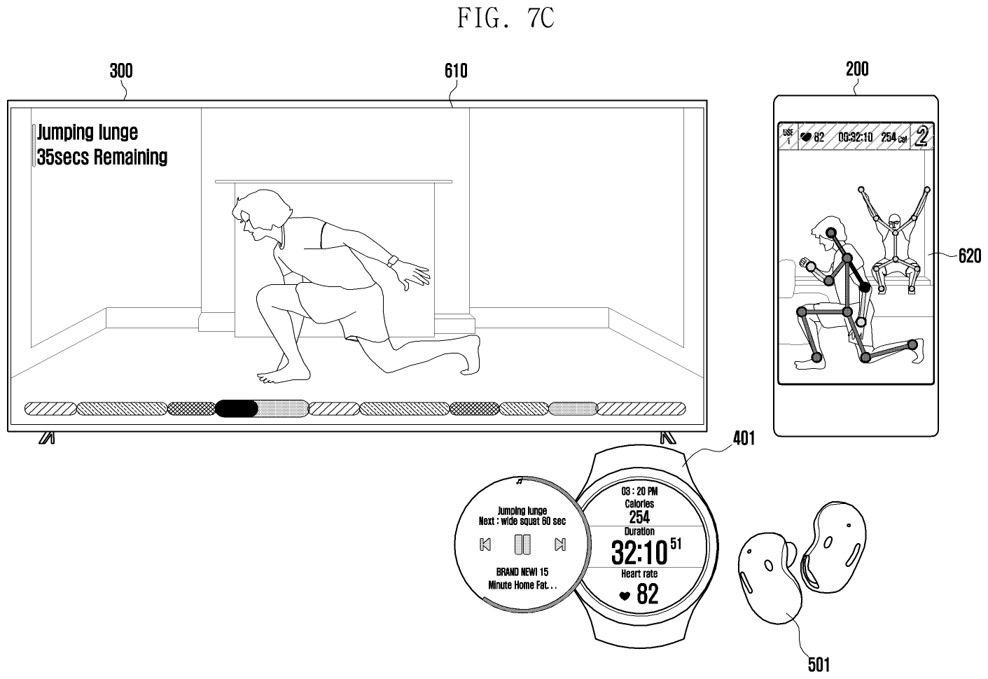

FIGS. 7A, 7B, and 7C are diagrams illustrating examples in which a processor controls external electronic devices to output various information from the external electronic devices, according to certain embodiments of the disclosure.

FIG. 7A is a diagram illustrating an example in which the processor controls to output various information to an external electronic device when there is one electronic device for photographing one user and external electronic devices worn by a plurality of users.

According to the embodiment of FIG. 7A, in response to detecting the presence of a plurality of users, the processor 250 may control the electronic device 200, the first external electronic device 300, the second external electronic device 401 of the first user, the third external electronic device 501 of the first user, the second external electronic device 402 of the second user, and/or the third external electronic device 502 of the second user to output various information.

The processor 250 may control the first external electronic device 300 to display the content screen 610 and/or the user screen 620 including the first user and biometric information and/or motion information of the first user, and to output the posture information of the object in an audio form (e.g., by providing the posture guide of the object by voice, “If you straighten your back and keep your knees from touching the floor, you can perform more effective jumping lunges.”).

The processor 250 may obtain the biometric information (e.g., heart rate 82, calorie consumption 254) and/or motion information (e.g., the number of operations twice) of the first user from the second external electronic device 401 of the first user, and may control the second external electronic device 401 of the first user so that the second external electronic device 401 of the first user displays the biometric information and/or motion information of the first user. The processor 250 may obtain the biometric information (e.g., heart rate 110, calorie consumption 293) and/or motion information (e.g., number of operations 0) of the second user from the second external electronic device 402 of the second user, and may control the second external electronic device 402 of the second user so that the second external electronic device 402 of the second user displays the biometric information and/or motion information of the second user.

The processor 250 may obtain a motion change input of the user from the second external electronic device 401 of the first user. When it is set to obtain the motion change input of the user from the second external electronic device 401 of the first user, the processor 250 may not change the operation even if there is an operation change input of the user from the second external electronic device 402 of the second user.

The processor 250 may analyze the user screen 620 including the first user and may generate first feedback information on the posture of the first user.

The processor 250 may control the third external electronic device 501 of the first user to output the first feedback information for the first user in an audio format (e.g., provide customized feedback information to the first user by voice, “Put your right hand on the floor and your left hand straight on your back”). The processor 250 may control the third external electronic device 502 of the second user to output a guide related to the biometric information and/or motion information of the second user in audio format (e.g., voice guidance based on biometric information, “You are maintaining a proper heart rate. To increase the intensity of your exercise, try a little harder”).

FIG. 7B is a diagram illustrating an example in which the processor controls to output various information to an external electronic device when there are a plurality of electronic devices for photographing a plurality of users and external electronic devices worn by the plurality of users (rather than for a single user, as in FIG. 7A).

According to the embodiment of FIG. 7B, in response detecting the presence of the plurality of users, the processor 250 may control an electronic device (not shown), a first external electronic device 300, a second external electronic device 401 of the first user, a third external electronic device 501 of the first user, a second external electronic device 402 of the second user, and a third external electronic device 502 of the second user to output various information.

The processor 250 may control the first external electronic device 300 to display the content screen 610, the first user screen 620 including an image of the first user captured by the electronic device of the first user and including biometric information and/or motion information of the first user, and/or the second user screen 630 including an image of the second user captured by the electronic device of the second user and including biometric information and/or motion information of the second user and to output the posture information of the object to be output in an audio format (e.g., provide posture guide voice of the object, “If you straighten your back and keep your knees from touching the floor, you can do more effective jumping lunges.”).

The processor 250 may obtain the biometric information (e.g., heart rate 82, calorie consumption 254) and/or motion information (e.g., the number of operations twice) of the first user from the second external electronic device 401 of the first user, and may control the second external electronic device 401 of the first user so that the second external electronic device 401 of the first user displays the biometric information and/or motion information of the first user. The processor 250 may obtain biometric information (e.g., heart rate 110, calorie consumption 293) and/or motion information (e.g., number of operations 0) of the second user from the second external electronic device 402 of the second user, and may control the second external electronic device 402 of the second user to display the biometric information and/or motion information of the second user.

The processor 250 may obtain a motion change input of a user from the second external electronic device 401 of the first user. When it is set to obtain the motion change input of the user from the second external electronic device 401 of the first user, the processor 250 may not change the operation even if there is an operation change input of the user from the second external electronic device 402 of the second user.

The processor 250 may analyze the first user screen 620 and/or the second user screen 630, and may generate first feedback information on the posture of the first user in the first user screen 620 and/or second feedback information on the posture of the second user in the second user screen 630.

The processor 250 may select an external electronic device for outputting the first feedback information and/or the second feedback information. According to an embodiment, the processor 250 may check the electronic device (e.g., the third external electronic device 501) worn by the first user corresponding to the first feedback information, and may determine whether to output the first feedback information to the checked electronic device as the electronic device. According to an embodiment, the processor 250 may check the electronic device (e.g., the third external electronic device 502) worn by the second user corresponding to the second feedback information, and may determine whether to output the second feedback information to the checked electronic device. For example, the processor 250 may analyze the user screen 620, and compare a part of the object included in the user screen 620 (e.g., the face of the first user) and identification information (e.g., the face of the first user) of the object previously stored in the memory (e.g., the memory 280 of FIG. 2). The processor 250 may transmit the feedback information related to an object matching the previously stored identification information of the object to an electronic device having a previously connected history (e.g., the third external electronic device 501 of the first user). The processor 250 may transmit the feedback information related to an object that does not match the previously stored identification information of the object (e.g., the second user) to an electronic device without a previous connection history (e.g., the third external electronic device 502 of the second user).

The processor 250 may select an external electronic device for outputting the first feedback information and/or the second feedback information. According to an embodiment, the processor 250 may check the electronic device (e.g., the third external electronic device 501) worn by the first user corresponding to the first feedback information, and may determine the checked electronic device as the electronic device to output the first feedback information. According to an embodiment, the processor 250 may check the electronic device (e.g., the third external electronic device 502) worn by the second user corresponding to the second feedback information, and may determine to output the second feedback information to the checked electronic device. For example, the processor 250 may transmit the first feedback information to an electronic device (e.g., a third external electronic device of the first user) having a history of being connected to the electronic device of the first user photographing the first user screen 620. The processor 250 may transmit the second feedback information to an electronic device having a history of being connected to the electronic device of the second user who photographed the second user screen 630 (e.g., the third external electronic device 502 of the second user).

The processor 250 may control the third external electronic device 501 of the first user to output the first feedback information for the first user in an audio format (e.g., provide customized feedback information for the first user by voice, “Put your right hand on the floor and your left hand straight on your back”), and may control the third external electronic device 502 of the second user to output the second feedback information for the second user in an audio format (e.g., provide customized feedback information for the second user in voice, “watch the video and take a correct posture”).

FIG. 7C is a diagram illustrating an example in which the processor controls an electronic device and/or an external electronic device to output various information when there is one electronic device for photographing a plurality of users and there is an external electronic device worn by one user.

According to the embodiment of FIG. 7C, the processor 250 may control the electronic device 200, the first external electronic device 300, the second external electronic device 401 of the first user, and the third external electronic device 501 of the first user to output various information in response to the fact that there are the plurality of users.

The processor 250 may control the first external electronic device 300 to display the content screen 610 and to output the posture information of the object in an audio form (e.g., provide the posture guide voice of the object, “If you straighten your back and keep your knees from touching the floor, you can perform a more effective jumping lunge.”).

The processor 250 may control the electronic device 200 to display the user screen 620 including the first user and the second user and including biometric information and/or motion information of the first user photographed by the electronic device 200.

The processor 250 may obtain biometric information (e.g., heart rate 82, calorie consumption 254) and/or motion information (e.g., the number of operations twice) of the first user from the second external electronic device 401 of the first user, and may control the second external electronic device 401 of first user so that the second external electronic device 401 of first user displays the biometric information and/or motion information of first user. A motion change input of the user may be obtained from the second external electronic device 401 of the first user.

The processor 250 may analyze the user screen 620 including the first user and/or the second user, and may generate first feedback information on the posture of the first user and/or second feedback information on the posture of the second user.

The processor 250 may select an external electronic device for outputting the first feedback information and/or the second feedback information.

According to an embodiment, the processor 250 may check the electronic device (e.g., the third external electronic device 501) worn by the first user corresponding to the first feedback information, and may determine the checked electronic device as the electronic device to output the first feedback information. For example, the processor 250 may analyze the user screen 620, and may compare a part of the object included in the user screen 620 (e.g., the face of the first user) and identification information (e.g., the face of the first user) of the object previously stored in a memory (e.g., the memory 280 of FIG. 2). The processor 250 may transmit the feedback information related to an object matching the previously stored identification information of the object to an electronic device (e.g., the third external electronic device 501 of the first user) having a previously connected history.

The processor 250 may control the third external electronic device 501 of the first user to output the first feedback information for the first user in an audio format (e.g., provide customized feedback information to the first user by voice, “Put your right hand on the floor and your left hand straight on your back”).

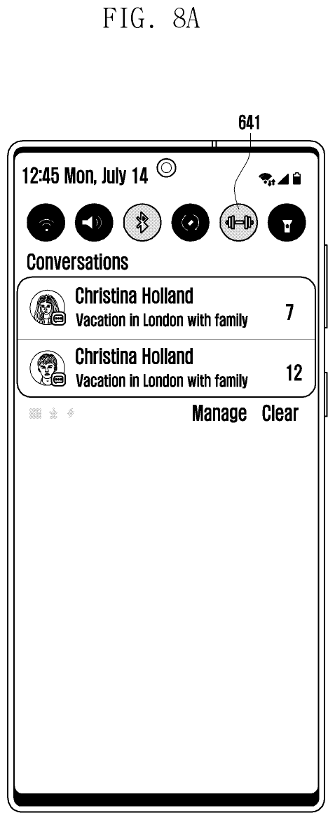

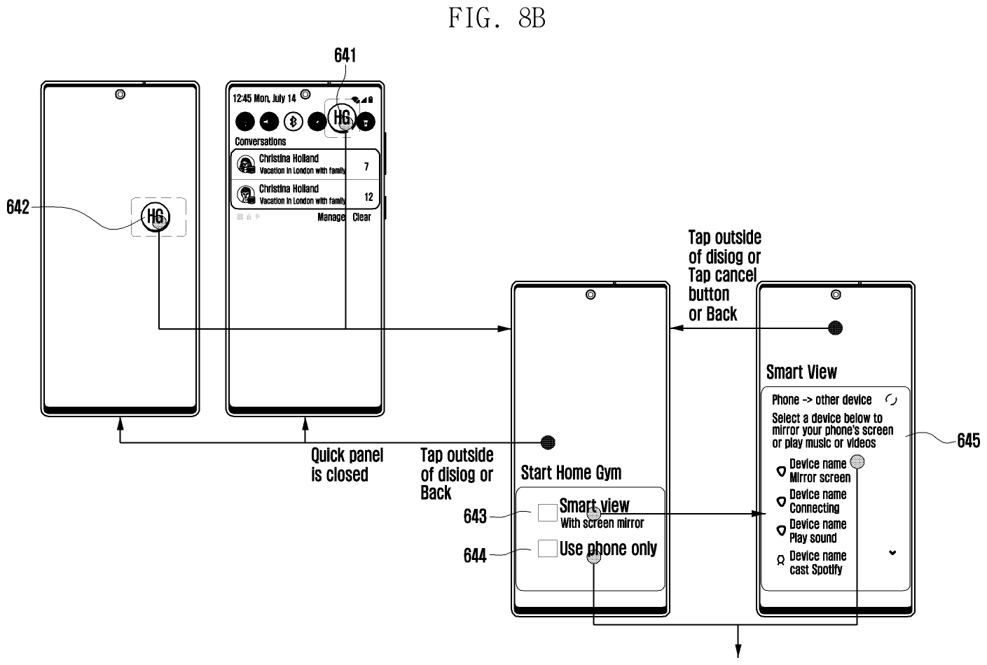

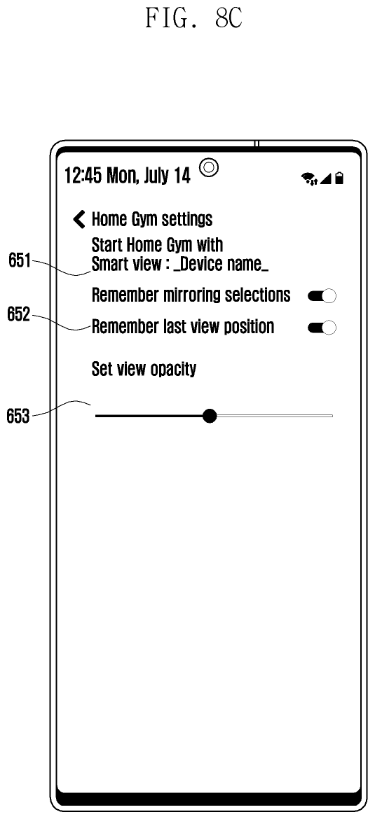

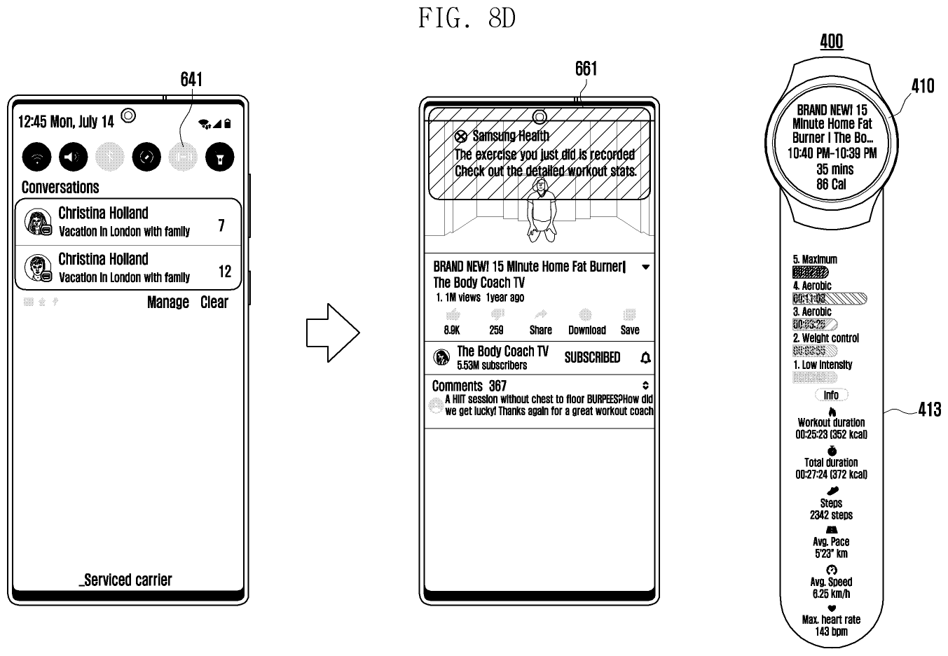

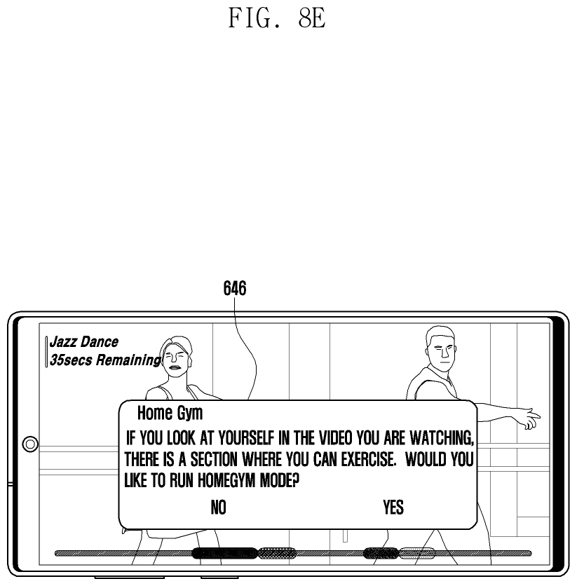

FIGS. 8A, 8B, 8C, 8D, and 8E are diagrams illustrating examples of a UI included in the electronic device according to certain embodiments disclosed herein.

The electronic device 200 may provide various types of user interfaces (UIs) for obtaining an input from a user in relation to the operation to provide feedback on the user's motion with respect to the operation of the content.

According to the embodiment of FIG. 8A, the processor (e.g., the processor 250 of FIG. 2) may display a UI related to activation of a mode that provides feedback on user movements on the display 220 in the form of an icon 641 within a menu bar.

According to an embodiment, the processor 250 may not initially display the menu bar on the display 220 in the standby state of the electronic device 200. Rather, the menu bar is selectively displayed responsive to a specific user input (e.g., scroll down or touch) requesting display of the same.

According to certain embodiments, the processor 250 may switch and display an activated state and/or an inactivated state of the icon 641 in response to a user's touch input. The processor 250 may execute a motion feedback mode in which a user's motion is analyzed with respect to an object motion in the content (e.g., trainer movements in a video) in response to activation of the icon 641. For example, the processor 250 may perform each operation of FIG. 3 when content is reproduced in response to the icon 641 being activated.

According to the embodiment of FIG. 8B, the processor (e.g., the processor 250 of FIG. 2) may display, on the display 220, a UI related to activation of a mode that provides a user's motion feedback on the operation of the content in the form of an icon 641 in the top bar and/or as a floating icon 642.

According to certain embodiments, the processor 250 may prompt the user for confirmation whether to execute a mirroring option related to displaying a related screen on an external electronic device in response to activation of the motion feedback mode, which feeds back the user's motion with respect to the motion of the object in the content. For example, the smart view option 643 may be an option related to displaying a content screen (e.g., the content screen 610 of FIG. 5A) and/or a user screen (e.g., the user screen 620 of FIG. 5C) on an external electronic device including a display (e.g., the first external electronic device 200 of FIG. 4). In response to the smart view option 643 being selected, the processor 250 may request the user to select 645 an external electronic device to output the content screen 610 and/or the user screen 620. In addition, the smartphone screen option 644 may be an option related to displaying the content screen 610 and/or the user screen 620 on a display (e.g., the electronic device 200 of FIG. 2) of the electronic device (e.g., the electronic device 200 of FIG. 2.

According to the embodiment of FIG. 8C, the processor 250 may provide a UI for configuration of a motion feedback mode that provides guidance for a user's motion with respect to an object motion in the content (e.g., a trainer within an exercise video). According to an embodiment, the processor 250 may provide a UI 651 related to whether or not the mirroring option is executed, a UI 652 related to the size, position, and ratio of the content screen 610 and/or the user screen 620, and a UI 653 related to transparency adjustment of the user screen 620.

According to the embodiment of FIG. 8D, the processor 250 may provide a user interface (UI) related to the end of the mode for providing user feedback on the operation of the content.

According to certain embodiments, the processor 250 may end the feedback mode in response to the icon 641 being switched to an inactive state by another touch input.

The processor 250 may provide a pop-up window 661 indicating the termination of the mode providing the motion feedback based on the content.

In response to terminating of the mode of providing the user's motion feedback on the operation of the content, the processor 250 may control to display user information 413 (e.g., operation time for each operation, total operation time, calories burned, average heart rate) related to the user performing an operation on the display 410 of the second external electronic device 400.

According to the embodiment of FIG. 8E, the processor 250 may display a UI related to activation of a mode providing user motion feedback with respect to object motion in the content on the display 220 (e.g., a trainer exercise video) in the form of a pop-up window 646.

According to an embodiment, the processor 250 may determine whether there is a section (e.g., a video segment) in which an object in the content performs a motion (e.g., an exercise or dance motion) in a designated field (e.g., exercise or dancing) while the content is being reproduced. The processor 250 may display on the display 220 a pop-up window 646 requesting a user input to confirm executing of the feedback mode providing user motion feedback based on analysis of motions of the object depicted in the content, when detecting a section in the video in which an object therein (e.g., a trainer or dance) performs a movement of the correct type (e.g., exercise, dancing).

An electronic device according to certain embodiments of the disclosure may include a display; camera; a memory for temporarily or non-temporarily storing content; and a processor operatively coupled to the display, the camera, and the memory, wherein the processor is configured to: analyze an image included in the content to generate posture information of an object included in the image; obtain an image including a first user through the camera while displaying the content; analyze the image including the first user to generate posture information of the first user; generate feedback information to be provided to the first user, based on a comparison result of the posture information of the object and the posture information of the first user; determine an external electronic device to output the feedback information, based on one of a property of the feedback information, a function of the external electronic device, the number of users, and a relationship between the user and the external electronic device; and transmit the feedback information to the determined external electronic device.

In the electronic device according to certain embodiments of the disclosure, the processor may check a similarity between the posture information of the object and the posture information of the user, based on the comparison result, and generate feedback information including information for classifying the user's posture into a matching region in which the similarity is equal to or greater than a first value, a similar region in which the similarity is less than the first value and greater than or equal to a second value, and/or a dissimilar region in which the similarity is less than the second value, based on the similarity.

In the electronic device according to certain embodiments of the disclosure, the processor may display a node corresponding to a part of the user's body and at least one line connecting the node on the image including the user, and display the matching region with a first color line, the similar region with a second color line, and the dissimilar region with a third color line.

In the electronic device according to certain embodiments of the disclosure, the processor may provide the posture information of the object in the form of audio, and control the external electronic device to output the posture information of the object to an external electronic device equipped with an audio output function.

In the electronic device according to certain embodiments of the disclosure, the processor may generate feedback information including information for guiding a posture in order to increase the similarity.

In the electronic device according to certain embodiments of the disclosure, the processor may discriminate and generates the posture information of the first user and the posture information of a second user, generate first feedback information in response to posture information of the first user, generate second feedback information in response to posture information of the second user, determine to output the first feedback information to a first external electronic device worn by the first user, and determine to output the second feedback information to a second external electronic device worn by the second user.

In the electronic device according to certain embodiments of the disclosure, the processor may implement the first feedback information and/or the second feedback information in a form of audio, determine to output the first feedback information to the first external electronic device equipped with an audio output function worn by the first user, and determine to output the second feedback information to the second external electronic device equipped with an audio output function worn by the second user.

In the electronic device according to certain embodiments of the disclosure, the processor may analyze the image included in the content to determine a first section related to a first motion, and analyze the image included in the content of the first section to generate motion information of the object included in the image.

In the electronic device according to certain embodiments of the disclosure, the processor may analyze the image included in the content of the first section to generate name information of the first motion, and control the external electronic device to display name information of the first motion on a display of the external electronic device.

In the electronic device according to certain embodiments of the disclosure, the processor may generate section information including a content playback time and/or a playback position of the first section, and control the external electronic device to display the section information of the first section on a display of the external electronic device.

A method of operating an electronic device according to certain embodiments of the disclosure may include an operation of analyzing an image included in temporarily or non-temporarily stored content to generate posture information of an object included in the image; an operation of acquiring an image including a first user through the camera while displaying the content; an operation of analyzing the image including the first user to generate posture information of the first user; an operation of generating feedback information to be provided to the first user, based on a comparison result of the posture information of the object and the posture information of the first user; an operation of determining an external electronic device to output the feedback information, based on one of a property of the feedback information, a function of the external electronic device, the number of users, and a relationship between the user and the external electronic device; and an operation of transmitting the feedback information to the determined external electronic device.

The method of operating an electronic device according to certain embodiments of the disclosure may include an operation of checking a similarity between the posture information of the object and the posture information of the user, based on the comparison result; and an operation of generating feedback information including information for classifying the user's posture into a matching region in which the similarity is equal to or greater than a first value, a similar region in which the similarity is less than the first value and greater than or equal to a second value, and/or a dissimilar region in which the similarity is less than the second value, based on the similarity.

The method of operating an electronic device according to certain embodiments of the disclosure may include an operation of displaying a node corresponding to a part of the user's body and at least one line connecting the node on the image including the user; and an operation of displaying the matching region with a first color line, the similar region with a second color line, and the dissimilar region with a third color line.

The method of operating an electronic device according to certain embodiments of the disclosure may include an operation of providing the posture information of the object in a form of audio; and an operation of controlling the external electronic device to output the posture information of the object to an external electronic device equipped with an audio output function.

The method of operating an electronic device according to certain embodiments of the disclosure may include an operation of generating feedback information including information for guiding a posture in order to increase the similarity.

The method of operating an electronic device according to certain embodiments of the disclosure may include an operation of discriminating and generating the posture information of the first user and the posture information of the second user; an operation of generating first feedback information in response to posture information of a first user; an operation of generating second feedback information in response to posture information of a second user; an operation of determining to output the first feedback information to a first external electronic device worn by the first user; and an operation of determining to output the second feedback information to a second external electronic device worn by the second user.

The method of operating an electronic device according to certain embodiments of the disclosure may include an operation of implementing the first feedback information and/or the second feedback information in the form of audio; an operation of determining to output the first feedback information to the first external electronic device equipped with an audio output function worn by the first user; and an operation of determining to output the second feedback information to the second external electronic device equipped with an audio output function worn by the second user.

The method of operating an electronic device according to certain embodiments of the disclosure may include an operation of analyzing the image included in the content to determine a first section related to the first motion; and an operation of analyzing an image included in the content of the first section to generate motion information of an object included in the image.

The method of operating an electronic device according to certain embodiments of the disclosure may include an operation of analyzing the image included in the content of the first section to generate name information of the first motion; and an operation of controlling the external electronic device to display name information of the first motion on a display of the external electronic device.

The method of operating an electronic device according to certain embodiments of the disclosure may include an operation of generating section information including a content playback time and/or a playback position of the first section; and an operation of controlling the external electronic device to display section information of the first section on a display of the external electronic device.

It should be appreciated that certain embodiments of the present disclosure and the terms used therein are not intended to limit the technological features set forth herein to particular embodiments and include various changes, equivalents, or replacements for a corresponding embodiment.

With regard to the description of the drawings, similar reference numerals may be used to refer to similar or related elements. It is to be understood that a singular form of a noun corresponding to an item may include one or more of the things, unless the relevant context clearly indicates otherwise.

As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

In addition, the embodiments disclosed in this document disclosed in the present specification and drawings are merely presented as specific examples to easily explain the technical content according to the embodiments disclosed in this document and help understanding of the embodiments disclosed in this document, It is not intended to limit the scope of the examples. Therefore, the scope of the certain embodiments disclosed in this document should be interpreted that, in addition to the embodiments disclosed herein, all changes or modifications derived from the technical ideas of the certain embodiments disclosed in this document are included in the scope of the certain embodiments disclosed in this document.

1. An electronic device, comprising:
a display;
a camera;
a memory configured to store content; and
a processor operatively coupled to the display, the camera, and the memory, wherein the processor is configured to:
analyze at least one image of the content to generate corrective posture information from an object depicted in the at least one image,
capture an image including a first user via the camera while displaying the content,
analyze the captured image including the first user to generate first posture information of the first user,
generate first feedback information, by comparing the corrective posture information to the first posture information,
select a first external electronic device for transmission of the first feedback information, based on one of a property of the first feedback information, a function of the first external electronic device, a number of users, and a stored relationship of the user to the first external electronic device, and
transmit the first feedback information to the selected first external electronic device.

2. The electronic device of claim 1, wherein the processor is further configured to:
determine similarities between preset body-regions depicted via the corrective posture information and the preset body-regions of the first user depicted via the first posture information,
wherein generating the first feedback information further includes:
classify each body-region of the first user into one of:
a matching region in which the similarity is equal to or greater than a first value,
a similar region in which the similarity is less than the first value and greater than or equal to a second value, and/or
a dissimilar region in which the similarity is less than the second value.

3. The electronic device of claim 2, wherein the processor is further configured to:
control the display to display each preset body-region using a plurality of nodes and a plurality of lines,
wherein lines within the matching region are depicted using a first color, lines within the similar region are depicted using a second color, and lines within the dissimilar region are depicted with a third color.

4. The electronic device of claim 1, wherein the processor is further configured to:
generates the corrective posture information of the object in audio format, and
wherein the first external electronic device is selected for transmission of the first feedback information based on inclusion of an audio output function in the first external electronic device.

5. The electronic device of claim 2, wherein the first feedback information further includes guidance prompting the first user to adjust a motion in order to increase the determined similarities.

6. The electronic device of claim 5, wherein the processor is further configured to:
generate second posture information for a second user captured by the camera,
generate second feedback information based on the second posture information of the second user,
select the first external electronic device for transmission of the first feedback information based on the stored relationship indicating that the first external electronic device is worn by the first user, and
select a second external electronic device for transmission of the second feedback information based on a stored relationship of the second user to the second external electronic device indicating that the second external electronic device is worn by the second user.

7. The electronic device of claim 6, wherein the first feedback information and/or the second feedback information includes information in an audio format,
wherein selecting the first external electronic device for transmission of the first feedback information is partly based the first external electronic device being equipped with an audio output function, and
wherein selecting the second external electronic device for transmission of the second feedback information is partly based the second external electronic device being equipped with the audio output function.

8. The electronic device of claim 1, wherein the content includes a video in which the at least one image is included, the processor further configured to:
determine that a first section of the video is related to a first motion, and
wherein analyzing the at least one image includes generating motion information of the object depicted in the at least one image included in the first section.

9. The electronic device of claim 8, wherein the processor is further configured to:
generate a name of the first motion by analyzing the at least one image included in the first section, and
control the external electronic device to display the generated name of the first motion on a display of the external electronic device.

10. The electronic device of claim 8, wherein the processor is further configured to:
generate section information for the first section associated with the first motion, including a playback time and/or a playback position of the first section, and
control the external electronic device to display the generated section information on the display of the external electronic device.

11. A method of operating an electronic device, the method comprising:
analyzing at least one image included in a stored content to generate corrective posture information of an object depicted in the at least one image;
capturing an image including a first user via a camera while displaying the stored content on a display;
analyzing the captured image to generate first posture information of the first user;
generating first feedback information by comparing the corrective posture information to the generated first posture information;
selecting a first external electronic device for transmission of the generated feedback information, based on one of a property of the first feedback information, a function of the first external electronic device, a number of users, and a stored relationship between the first user to the first external electronic device; and
transmitting the generated feedback information to the selected external electronic device.

12. The method of claim 11, further comprising:
determining similarities between preset body-regions depicted via the corrective posture information and the preset body-regions of the first user depicted via the first posture information,
wherein generating the first feedback information further includes:
classifying each body-region of the first user into one of:
a matching region in which the similarity is equal to or greater than a first value,
a similar region in which the similarity is less than the first value and greater than or equal to a second value, and/or
a dissimilar region in which the similarity is less than the second value.

13. The method of claim 12, further comprising:
display each preset body-region using a plurality of nodes and a plurality of lines on the display,
wherein lines within the matching region are depicted using a first color, lines within the similar region are depicted using a second color, and lines within the dissimilar region are depicted with a third color.

14. The method of claim 11, further comprising:
generating the corrective posture information of the object in audio format, and
wherein the first external electronic device is selected for transmission of the first feedback information based on inclusion of an audio output function in the first external electronic device.

15. The method of claim 12, wherein the first feedback information further includes guidance prompting the first user to adjust a motion in order to increase the determined similarities.

16. The method of claim 15, further comprising:
generating second posture information for a second user captured by the camera;
generating second feedback information based on the second posture information of the second use;
selecting the first external electronic device for transmission of the first feedback information based on the stored relationship indicating that the first external electronic device is worn by the first user; and
selecting a second external electronic device for transmission of the second feedback information based on a stored relationship of the second user to the second external electronic device indicating that the second external electronic device is worn by the second user.

17. The method of claim 16, wherein the first feedback information and/or the second feedback information includes information in an audio format,
wherein selecting the first external electronic device for transmission of the first feedback information is partly based the first external electronic device being equipped with an audio output function, and
wherein selecting the second external electronic device for transmission of the second feedback information is partly based the second external electronic device being equipped with the audio output function.

18. The method of claim 11, wherein the content includes a video in which the at least one image is included, the method further comprising:
determining that a first section of the video is related to a first motion, and
wherein analyzing the at least one image includes generating motion information of the object depicted in the at least one image included in the first section.

19. The method of claim 18, further comprising:
generating a name of the first motion by analyzing the at least one image included in the first section, and
controlling the external electronic device to display the generated name of the first motion on a display of the external electronic device.

20. The method of claim 18, further comprising:
generating section information for the first section associated with the first motion, including a playback time and/or a playback position of the first section, and
controlling the external electronic device to display the generated section information on the display of the external electronic device.
